SEQUENCE LISTING (16 sequences)

SEQ ID NO: 1 - protein, 182 aa, Bacillus cibi [sequence omitted]

SEQ ID NO: 2 - protein, 610 aa, Trichoderma harzianum
APANVAIRSLEERASSADRLVFCHFMIGIVGDRGSSADYDDDMQRAKAAGIDAFALNIGVDGYTDQQLGYAYDSADRNGMKVFISFDFNWWSPGNAVGVGQKIAQYASRPAQLYVDNRPFASSFAGDGLDVNALRSAAGSNVYFVPNFHPGQSSPSNIDGALNWMAWDNDGNNKAPKPGQTVTVADGDNAYKNWLGGKPYLAPVSPWFFTHFGPEVSYSKNWVFPGGPLIYNRWQQVLQQGFPMVEIVTWNDYGESHYVGPLKSKHFDDGNSKWVNDMPHDGFLDLSKPFIAAYKNRDTDISKYVQNEQLVYWYRRNLKALDCDATDTTSNRPANNGSGNYFMGRPDGWQTMDDTVYVAALLKTAGSVTVTSGGTTQTFQANAGANLFQIPASIGQQKFALTRNGQTVFSGTSLMDITNVCSCGIYNFNPYVGTIPAGFDDPLQADGLFSLTIGLHVTTCQAKPSLGTNPPVTSGPVSSLPASSTTRASSPPVSSTRVSSPPVSSPPVSRTSSPPPPPASSTPPSGQVCVAGTVADGESGNYIGLCQFSCNYGYCPPGPCKCTAFGAPISPPASNGRNGCPLPGEGDGYLGLCSFSCNHNYCPPTACQYC

SEQ ID NO: 3 - protein, 433 aa, Aspergillus aculeatus
QQAGTSTAETHPALTWQKCTAAGSCTEQSASVVIDSNWRWVHSVEGYTNCYTGNTWDATLCPDDETCASNCALDGADYEGTYGVTTSGDALTLKFVTGSNVGSRLYLMDTSDEKYQTFDLLDAEFTFDVDVSNLPCGLNGALYFTAMDADGGVSRFPANKAGAKYGTGYCDSQCPRDLKFIDGQANVEGWTPSTSNSNSGVGNHGSCCPEMDIWEANKISTAFTPHPCDSVTQVMCEGDACGGTYSSSRYAGTCDPDGCDFNTYRMGNKTFFGPGKTVDSNSKMTVVTQFIKGSSGSLSEIRRYYVQNGIVIANAASDISGVSGNSITSDFCTAQKTAFGDEDIFGDHDGLTKMGDALSSMVLIMSLWDDSYSSMEWLDSHYPTNATVTDPGVARGTCETGEGDPKTVEAAHPDASVTFSNIKFGPINSTFSA

SEQ ID NO: 4 - protein, 424 aa, Humicola insolens
LKEVFAHVITGNMPEMQVSEWEADMRLAKEAHIDGFVLNIASQDASNNNSIARAFEAAERVGNFKLFFSFDYEAQGPWPADRVANLLAEYIWRAPYFKHQGNRPLVSTFEGPNNANDWNTIKQLVGNIFFIPDWSSRAPNEAIGLGGGVADGLFNWDAWPEGKNDKTTASDQAFRAALGGKPYMMPVSPWFFTNLPGWNKNWLWKGDGLWDLRWRQVAEVKPEYVQILTWNDFGESHYIGPVHEKQLALFNYGQAPINYARNMPHDAWRKFLPFCIDVYKNDGRVPANVGQEGVVAYYRIAPARACPSGGTTGNNPNHGQTVLPPEEVLSDNVFFAALLNTNRGVEVTVSIGGLEQKASVFTLVPAAGAGTPGVYTGSVPFGDRTGDVVVTVKRDGNTIATARGSRPISRTCENNVQNWNAVVA

SEQ ID NO: 5 - protein, 598 aa, Trichoderma reesei
APANVAIRSLEERASSADRLVFCHFMIGICGDRTSSTDYDDDMQRAKAAGIDAFALNIGVDGYTDQQLNFAYDAADRAGMKVFISFDFNWWSPGNAAGVGQKIAQYASRPAQLYVDNRPFASSFAGDGLDVNTLRNAAGSNVYFVPNFHPGQSSPSTIDGALNWMAWDNDGNNKAPKPGQNVTVADGDNSYRSWLAGKPYLAPVSPWFFTHFGPEVSYSKNWVFPGGSLWYDRWQDVLRQGFEMVEIVTWNDYGESHYTGPLESRHYDDGNSKWTNDMPHDGFLDLAKPFIAAYKNRDTDVAPYIQNEQLIYWYRRNLKGLDCDATDTTSNRPANNGSGNYFMGRPDGWQTMDDTVYVVALLKSAGTVTVTSGGATQTFQGTAGANLFEVPANLGQQKFALSRNGQTVFSSTSLMDITNVCPCGIYNFNPYVGTVPAGFDDPLGPDGLASLTIGLHVTTCQAKPSLGTNPPITSGPGSSVPVSTPPGSTTRFSSTPVSSRSSSSTPPVSTPPPGQVCVAGTVADGQSGNYIGLCNFSCNFGYCPPGPCKCTAYGAPINPPATNGRNGCPLPGEDDSYLGLCSFSCNHNYCPPTACQYC

SEQ ID NO: 6 - protein, 601 aa, Talaromyces bacillisporus
LPGSDRSLTARQSTDRLVFAHFMIGITSDRHSASDFDADMQRAKAYGIDAFALNIGVDPYTDEELGYAYDSAANNGMKVFISFDFNWWNTGQATAIGQKIAQYASKPAQLMVDNKVFVSSFAGDGVDVAGIRSAAGMDLFFAPNFHPSYGTDLSSVDGLLNWMAWPNNGNNKAPTAGANVTVQDGDEQYINALGGKAYIAPASPWFSTHFGPEVSYSKNWVFPSDLLWYNRWQDLLTLGPRFIEIVTWNDYGESHYIGPLDSPHTDDGASKWVNDMPHDGWLDMAKPFIAAFKDGATSIDSYITDDQLIYWYRPTPRGVDCDATDTCMVSAPNASGNYFIGRPNGWETMEDSVFVVSLFKSAATITVNSGGNVQTFNAPSGASSFEVPMGVGAQTFSVTRNGQTVFEGTSLKQIINGCVCGLYNFNAYVGSIPAGFADPLQPDGLASLTQGLHVTTCSATPSLGTAPPTSATVTSTVSTVTTTTSTPATTASSTSTTVTSTATTKTTTTTSIATTTQPSSSSVCIGGTGPGNYVGLCNFCCQYGYCPPGPCTCTQYGAPVPTPPTNGRDGRPLPNEDDSYLGLCSFACNHGYCPDTACMYV

SEQ ID NO: 7 - protein, 608 aa, Talaromyces apiculatus
HPGHERSLTERASTDRLVFAHFMIGITSDRTSSADYDADMQRAKSYGIDAFALNIGVDPYTDQQLGFAYASAANNDMKVFISFDFNWWHTSQATAIGQKIAQYASLPAQLKVDDKVFVSSFAGDGVDIAAMRSAAGTELFFAPNFHPSYGTDLSNVDGLLNWMAWPNNGNNKAPDATGNVTVEQGDQQYVSALAGKAYIAPASPWFSTHFGPEVSYSKNWVFPSDLLWYNRWQELLTLGPRFIEIVTWNDYGESHYIGPLDSPHTDDGASKWVNDMPHDGWLDLAKPFIAAFKAGSTTVDSYITSDELIYWYRPTPKDVDCDATDTCMDPNASNSSGNYFIGRPNGYQTMEDSVFVVSLFTAAAEITVTSGGNSQTFQASAGANAFQVPMGVGTQTFSVTRNGATVFQGTSLKQIIDGCVCGLYNFNAYVGTLPAGFSDPLGPAGLASLVQGLHVSTCAATPSLGTAPPVTTTSVPVTTTRSTTTTTSTSSTKTTSTTTTTPTITTTSTTKTTTSTSTTSSTSTSTSGTVCIAGTGPGNYVGLCSFCCNYGYCPPGPCTCTAYGAPVPTPPVTGTVGLPLSNEDDSYLGLCSFACNHGYCPSTACRVA

SEQ ID NO: 8 - protein, 742 aa, Streptomyces yanii
AGVPAPSPLAVPGRGATVPFKEQEAEYATTNGTLIGPNRLYGTLPSEASGRQAVTLDAVGEYVEFTLTAPANAMSFRYSLPDSPDGTGRDASIDVRVGDGAPKSVPVTSKYGWYYGGYPFNNNPGDTNPHHFYDEARTMFGSTLPIGTKVRLQVSSTAASPTFTIDLADFEQVGAPVGRPSGALDVVSDFGADPTGATDSTGRIQAAVDAGRAQGKEVYIPQGTFQVRDHIVVDKVTLRGAGPWYSVLTGRDPSNRSKAVGVYGKYAADGGSSNVTLKDFAIIGDIRERVDNDQVNAIGGAMSNSVVDNIWMQHTKCGAWMDGPMDNFTVKNSRILDQTADGVNFHYGVTNSTVTNTFVRNTGDDGLAMWAENVPNVKNKFTFNTVILPILANNIVTYGGKDITISDNVMSDTITNGGGLHVANRYPGVNSGQGTAVSGITTAARNTLIRTGNNDFNWRFGVGAVWFSGLNEPINATINITDTEILDSSYAAIHLIEGATNGLHFDNVRIDGAGTYALQIQAPGTATFTDVVATHIAQSNPIHNCVGSGFQITRGSGNSGWYSDPPACTGTWPDPVWTNGGVPGGGTDPTDPPTDPPTDPPADPGNIALGRPVTESGHADVYGAANAVDGNANSYWESTNHAFPQTITVDLGAPKAVKRVVLKLPPATAWATRTQTLSVSGSADNSSYSSLKGSAGYVFDPADGNTATITLPGTSARYLRLTFTANTGWPAGQLSELEAYTS

SEQ ID NO: 9 - protein, 771 aa, Streptomyces alni
AGVPAPSPVGIAGRGANVPFKELEAEYANTNGSLIGPDRYYGHLPSEASGRQAVTLNSVGQYVEFTLTAPANAMSLRYSLPDTSDGKGRDASLDVQVNGSSVKTLPVTSKYSWYYGGYPFNNNPGDSNPHHFYDEARTMFPATYPIGTKIRVQVTSTAQSPSFTIDLADFEQVAGAIGQPSGSINAVTDYGVDPTGATDSTAKMQAAVNAGQAQHKVVYVPQGTYTLYDHVIVDGVTVQGAGPWYTVFGGRDPVQRNRAAGFYGKYVAGGGYGGAIRPQEAGGPSQNVTLKDFAIIGDIQERVDDDQVNGIGGAMSNSTIDDLWIQHVKVGAWMDGPMDHFTIKNSRILDTTADGVNFHSGVTNSTVTNTFVRNVGDDGLASWPEKAPNVSDSFDHNTVVLPILANNIVTYGGKDFNLSDNVMADTITNGGGLHIANRYPGVNGGGPNGVSGTITAARNTLIRTGNSDYNWQFGVGAIWFDGLQGKVDANIQISDTDILDSSYEAIQSIESGVTGATFKNINIDGAGTYAIQAQAAADLTFINVKAKNIAQSATPIHNCVGTALNIIDGGGNSGLTGQTCTGTWPTPVWTNGGVPTGGGGSTTTPPTTPPTTPPTTPPTTPPTTPPTTPPTTPPANPNLAQGHAASDTGHADVYVAGNTVDGNVNTYWESTNSAFPQSLTVDLGAAKTVGRLVLKLPTNWGTRTETLSVLGSTDNSSYSTLKASAGYTFTLGTDTATVTFTPTTARYLRLTFTGNTGWPAGQLSELEAYAS

SEQ ID NO: 10 - protein, 715 aa, Oribacterium sinus
ETRTELWAKESQSYGAKLPYLRYEAEEGVGRDAVLKHSTAYDEVEMEASNQSYVQLTKEDSSLRFTVKKAANAMTLRFTMPEDASGELEISVERNGELLGKKTVELDNSSAWQYVKENDVFDENIADSHSRFRFDERHFLLENDNKELMELEAGDVLTIRRTDKKADELGIDFIELEQVPEAKGAPSNSISITDAPYSAVPNDGQDDSQAFLDALKDADAENKTLYIPEGTFDFDQKLVLSATDMRITGAGIWYTRLHFTSEEQAGGGIEFLDSSSNVEMDNLYMDSELKSRFHQEANYKGIAGVLGENSKLHDLWLEHFECGIWVGDYVEADKMKYTKNLTVSNSRIRDNFADGVNFAQGTRNSKVQNSDIRGNGDDGLATFASKAIVKIKENVNGVEQVRYIHTESKPAENNAFLNNTVELTWRASGIALHGGANHHIEGNLVKDITSGPGLRVSTVFPGYNFDDNQNISIKRNLLIQTGTDNDFYGNALASIHFEKLYGDMKKITVADNCLINSPHGKYSGNFYIPEEGTTEITLRNNEEKSIDVEPMLAEEEKNFAPLSEEEKLVEEQKKAEEQKKAEELKKAAEQAEQAKHEGEQPGYDGALNIELHDQEEIPETANFRLYWFSGETEENGRTVRYWVKKLEGIHLDESMEYSLEDGTKVFNFTPDDIPEYIPISGTAFVEDYYYEGEDWIRWESPEGVEYVKIRYWSLK

SEQ ID NO: 11 - protein, 743 aa, Streptomyces galbus
ATGVPAPSPVGIPGRGADVPFKETEAEYASTNGTLIGPDRLYGHLPSEASGRQAVTLDAVGEYVEFTLTAPANAMSLRYSLPDSADGRGRNATLDVQVDGKSVKTLPVTSKYGWFYGGYPFNNNPGDSNPHHFYDEARTMFATTYPAGTKLRVQVTSTVQSPSFTIDLADFEQVAAPIAKPAGAIDAVADYGADPTGATDTTARIQAAVDAGHAQGRAVYVPKGDYTLYGHVIVDGVTVQGAGPWYTVFGGRDPVDRSRAAGFYGKYVTNGGPSRNVTLKDFAIIGDIQERNDDAQVNALGGAMSNSTVDHLWIQHVKVGAWMDGPMDHFTIKNSRILDTTADGVNFHTGVTDSTVTNTFVRNTGDDGLASWPQDKPNVNDSFDHNTVVLPILANNIVTYGGKDFNLSDNVMADTITNGGGLHIANRYPGVSASAGTAVAGTITAARNTLIRTGNSDYNWNFGVGAIWFDGLQGKVDADIRISDTDILDSSYEAIQSIESGVTGVTMKNVNIDGAGTYAIQAQAAATMTFINVTAKHIGQAATPIHSCVGSDLRITDGGGNSGWNTGQTCTGTWPAPIWTNSGVPSGGGTTPPTTPPTTPPAPSDVNLAKGRPVVDTGHADVYTAGNTVDGNANTYWESSNNAFPQSLTVDLGSARTVGRLVLKLPPATAWSTRTQTLSVLGSTDNSGWTTLKSSAGYTFNPSSGNSVTLTLNSATTRYLRLTFTGNTGWPAGQLSELEAYSS

SEQ ID NO: 12 - protein, 689 aa, Streptomyces glomeratus
DAGASLPFTSVEAESATTNGTKIGPDYTQGSLASEASGRQAVRLTSGQRVEFTVPRAANAVNLAYSVPDGQSGTLNVYVNGTRLTKTLAVTSKYSYVDTGWIPGARTHHFFDNARLLLGQNVQPGDRIAFESTGTQVTVDVADFEQVAAAASQPAGSVSVTSKGADPSGQGDSTQAFRDAIAAAQGGVVWIPPGDYRLTSSLSGVQNVTLQGAGSWWSVVHTSRFIDQSGSSGNVHIKDFAVLGEVTERVDSSPDNFVNGSLGPNSSVSGMWIQHLKVGLWLTGNNDNLVVENNRILDTTADGLNLNGTAKGVRVRNNFLRNQGDDALAMWSLYSPDTDSSFENNTISQPNLANGIAIYGGTDLAVRNNLISDTNALGSGIAISNQKFLDPFSPLAGTITVDGNTLVRTGAMNPNWNHPMGALRVDSYDSAINATVNITNTTITDSPYSAFEFVSGGGQGYPVRNVTVDGATVRNTGTVVVQAEAPGAATLRNVTATSVGSAGVYNCPYPANSGGFALTDGGGNSGWNSTWSDCSTWPQPGQGNPPPDPGRNLAKGRPATASGSQDVYTPGKAVDGDANSYWESTNNAFPQSFTVDLGSSQAVRRLVLKLPPLSAWQARAQTLSVLGSTDGSGYTTVVGSADYRFDPATGNTVTVTLPGGTNLRYLRLNVTANTGWPAAQFSEVEAYLS

SEQ ID NO: 13 - protein, 631 aa, Tetrasphaera sp.
GEPTAPATTAPKITRTALDPSLVQGRGATVPFVEQEAENAATDGQVLAPGREAYTLNAEASGRSAVRLNQPGQYVEFTLTGDTNAFTLRYSIPDAPAGGGITSPLTVTVDGHHDSTMTLTSQYSWLYGMYPFSNDPNVDPNPGWWKPEPDPVTKPFRPNHFYDEQRVLLGKTYHAGDRIRFTVPQGAAAAWTVLDVADFELVPKPLVKPHGALSVLDFGADPTGARESSDAIDATIAAAKTRGLTVFIPKGTYQVNRHIVVDDVTVQGAGSWWTIIKGHLIPLATPAPDKSTHSAPGFYGKYAADGGSHNVHLSDFAIESDVRERIDTDQVNGIGGALGGGSTVERLYIHHTKAGVWLDGPLSGLTLKDNVIADQIADGINLHQGVSDVRATNNFLRNTGDDGMAMWSENVNGPQDADHDNVFDHNTVQTPVLANDIAIYGGHDNTVSDNLVADPIREGSTLHAGARFNEVPFSGTTTFTRNTTVRGGPRDLNWDLGLGSVWFYLLDNSMHGQIYVTDSSILDSTYNAFMFVADWPVKDTYDITNVHISNIKVDGTGTNVVSARAAGWATFDNVVARNVGDPFVNDCGTFHFTGTPEFDVRLGAGNSGWTQWGSWCQDRPTVVPPPPPSAW

SEQ ID NO: 14 - protein, 773 aa, Kitasatospora phosalacinea
AAVPTLSPLAVPNRGASVPFVEQEAEYAATNGTVVGPDRQYGHLASEASGRQAVTLNAVGQYVEFTLTAPTNSIDFRYSLPDSADGKGRDASMDLLVNGQKLKSVDVTSKYSWYYGGYPFNNNPGDSNPHHFFDETRTLLGSTYPAGTKVRLQVSSTAQSPTFTVDLADFEKVADPIAKPTGAIDVVADYGADPTGATDSTAKFQAAVNAGAAQGRTVYIPQGAFTLYDHVVVDKVTLAGAGPWYSVLGGRDPINRNRAAGIYGKYVAGGGYGGGIRPQEAGGPSQNVTLKDFAIIGDVRERVDDDQVNGLGGAMSNSTVDNLWIQHTKVGAWMDGPMDHFTIKNSRILDQTADGVNFHTGVTNSTVTNTFLRNTGDDGLASWPERLANVNDSFTHNTVVLPILANNIVTYGGKDFTISDNVMADTITNGGGLHIANRYPGVTSGNGTAVSGTITAARNTLIRAGNSDYNWQFGVGAIWFDGLNEPITGATINISDTDILDSSYEAIQTIEGGVTGVNLTNVNIDGAGTYAIQAQANAQMKFTNVKAKNIAQAATPIHNCVGTGFQITDGGGNSGLSGSTCTGQWPAPVWTYNGVPVGGGNGGTSSPSPSPSPSTSVPASPSPSAPPSSTCPSGTGNLAQGRPATASGTSQTYGPGNAVDGDANTYWESANNAFPQWIQVDLGCARNLTKAVLKLPPATAWATRTQTVTVQGSTDGTTFTTLVGATGYTFDPATGNTVTVNLPANSTRYLRLTVTGNTGWPAGQLSELQAFTS

SEQ ID NO: 15 - protein, 835 aa, Aspergillus aculeatus
AASQAYTWKNVVTGGGGGFTPGIVFNPSAKGVAYARTDIGGAYRLNSDDTWTPLMDWVGNDTWHDWGIDALATDPVDTDRLYVAVGMYTNEWDPNVGSILRSTDQGDTWTETKLPFKVGGNMPGRGMGERLAVDPNKNSILYFGARSGHGLWKSTDYGATWSNVTSFTWTGTYFQDSSSTYTSDPVGIAWVTFDSTSGSSGSATPRIFVGVADAGKSVFKSEDAGATWAWVSGEPQYGFLPHKGVLSPEEKTLYISYANGAGPYDGTNGTVHKYNITSGVWTDISPTSLASTYYGYGGLSVDLQVPGTLMVAALNCWWPDELIWRSTDSGATWSPIWEWNGYPSINYYYSYDISNAPWIQDTTSTDQFPVRVGWMVEALAIDPFDSNHWLYGTGLTVYGGHDLTNWDSKHNVTVKSLAVGIEEMAVLGLITPPGGPALLSAVGDDGGFYHSNLDAAPNQAFHTPTYGTTNGIDYAGNKPSNIVRSGASDDYPTLALSSNFGSTWYADYAASTSTGTGAVALSADGDTVLLMSSTSGALVSKSQGTLTAVSSLPSGAVIASDKSNNTVFYGGSAGAIYVSKNTATSFTKTVTLGSSTTVNAIRAHPSVAGDVWASTDKGLWHSTDYGSTFTQIGSGVTAGWSFGFGKASSTGSYVVIYGFFTIDGAAGLFKSEDAGTNWQLISDASHGFGSASANVVNGDLQTYGRVFVGTNGRGIFYGNPSGSLPSATATATATTSKTSTTVSTTLKTTTSASTSSSSTTMKTTTSSSSTTSKASSTTTTTTKTTGTTTTTSSGSTATASAYAQCGGSGWTGATVCVSGYKCTYSNDFYSQCVPS

SEQ ID NO: 16 - protein, 396 aa, Aspergillus aculeatus
APQAKRSSNFLWLGSNESGAEFGQANIPGVLGTDYIWPDASKIQVLVNAGMNIFRVPFMMERLIPTSLTGTADATYLADLKATIESITSLGAYAVVDPHNFGRYYGNIITSTSDFAAFWTTVAKVFASNDHVIFDTNNEYHDEDQTLVFNLNQAAINAIRAAGATSQYIFVEGNSYSGAWTWTTYNTNLVNLTDPSDKIIYEMHQYLDSDGSGTSADCVSTTIGAERVAAATTWLRQNGKRAVLGEFAGGANSQCLTAVKGMLDALSAASDVWLGALWWSAGPWWGTYIFNMEPTTGTAYTYYLSLLQSYVPGGSSGTTTTTTAVATTTTTKASTTSTATTTKTTTSTKTTTAATSTSTAVAPLYGQCGGIGWTGATTCASGSTCTKQNDYYSQCL

(19) United States
(12) Patent Application Publication
(10) Pub. No.: US 2026/0151318 A1
(43) Pub. Date: Jun. 4, 2026

(54) ORAL CARE COMPOSITION COMPRISING ENZYMES

(71) Applicant: Novozymes A/S, Bagsvaerd (DK)

(72) Inventors: Manish Kumar TIWARI, Charlottenlund (DK); Lorena González PALMÉN, Akarp (SE); Dorotea Raventos SEGURA, Rungsted (DK); Mette Rose JOERGENSEN, Birkeroed (DK)

(73) Assignee: Novozymes A/S, Bagsvaerd (DK)

(21) Appl. No.: 18/719,284

(22) PCT Filed: Dec. 13, 2022
(86) PCT No.: PCT/EP2022/085665

§ 371 Date: Jun. 13, 2024

(30) Foreign Application Priority Data
Dec. 16, 2021 (DK) PA202101210
Jun. 30, 2022 (EP) 22182251.3

(51) Int. Cl. A61K 8/66; A61Q 11/00

(52) U.S. Cl. CPC A61K 8/66; A61Q 11/00; C12Y 302/01006; C12Y 302/01059

(57) ABSTRACT

The present invention relates to an enzymatic oral care composition comprising a DNase, a mutanase, a beta-glucanase, and at least one oral care ingredient. The invention further relates to the use of said composition as a medicament, use of said composition in treatment of oral disease, methods of treatment comprising administering said composition to a human subject, methods of removing biofilm comprising contacting an object with said composition, and kits of parts comprising said composition.

### REFERENCE TO SEQUENCE LISTING

This application contains a sequence listing in computer readable form created on Jan. 14, 2026, named Corrected2_SQ_ST26.xml and 24,491 bytes in size, which is incorporated herein by reference.

### FIELD OF THE INVENTION

The present invention relates to an enzymatic oral care composition comprising a DNase, a mutanase, a beta-glucanase, and at least one oral care ingredient. The invention further relates to the use of said composition as a medicament, use of said composition in treatment of oral disease, methods of treatment comprising administering said composition to a human subject, methods of removing biofilm comprising contacting an object with said composition, and kits of parts comprising said composition.

### BACKGROUND OF THE INVENTION

Biofilms are communities of bacteria that are found on solid surfaces in many different environments, including surfaces of the oral cavity. Oral biofilm, or dental plague, contains many of the bacteria that are associated with oral health issues such as oral malodor, demineralization, dental caries, tooth decay, potential loss of teeth and gum disease (gingivitis and periodontitis).

The formation of oral biofilm occurs in three stages known as the lag phase, growth phase, and steady state, respectively. In the lag phase, glycoproteins from saliva bind to an oral surface such as teeth and create a structure termed the pellicle that functions as attachment site for bacteria. In the growth phase, co-aggregation occurs, i.e., secondary bacterial colonizers attach to the primary bacterial colonizers, causing the diversity of the biofilm to increase and the biofilm to grow and mature. In the steady state, the biofilm growth slows down and eventually stops. This stage-based formation cycle causes biofilms to exist in several consecutive layers, which makes physical abrasion of biofilm more difficult.

Within a biofilm, the residing bacterial cells are distributed in an extracellular polymeric matrix that consists primarily of water, proteins, exopolysaccharides, lipopolysaccharides, lipids, surfactants, and extracellular DNA, with exopolysaccharides occupying a major fraction of the dry weight of biofilm (H. C. Flemming, and J. Wingender (2010), Nat. Rev. Microbiol. 8, 623-633). The exopolysaccharides are mainly glucose and fructose homopolymers, including 1,3-α-D-glucans, 1,4-α-D-glucans, 1,6-α-D-glucans and 2,6-β-D-fructans. These polysaccharides are synthesized from ingested sucrose by glucosyltransferases and fructosyltransferases secreted by oral bacteria such as Streptococcus spp., Lactobacillus spp., and Actinomyces spp.). Mutans and dextrans are particularly important glucans in the formation of dental plaque. Mutans have a highly branched structure with main chains composed of glucose molecules linked with 1,3-α-glycosidic linkages and 1,6-α-glycosidic linkages in their side chains. Dextrans are also high molecular weight polymers of glucose containing numerous consecutive 1,6-α-linkages in their backbone and side chains, which begin from a 1,3-α-linkage (M. Pleszczynska et al. (2016), Biotechnol. Appl. Biochem. 64(3), 337-346).

Because of the increased resistance to anti-microbial agents as well as the mechanical properties of biofilm, many current oral care products are rather inefficient in addressing biofilm formation and alleviating the associated oral health issues. The main focus for biofilm removal has been on mechanical abrasion. However, this approach is difficult due to the multilayered nature of biofilms and is further compromised by the fact that mechanical removal of biofilm, e.g., by brushing the teeth, expands and deepens the areas in the oral cavity where biofilms attach and expand, thus potentially increasing the severity of the problem rather than reducing it.

In view of the important role of biofilm in oral disease, there is a need in the art for oral care compositions that can effectively target oral biofilm. WO 1997/38669 (Novozymes) describes oral care compositions comprising a mutanase and a dextranase, WO 1998/57653 (Novozymes) provides oral care compositions comprising a dextranase and a pullulanase, and WO 2020/099490 (Novozymes) describes oral care compositions comprising a mutanase and a DNase. However, there is a still need for further and improved oral care compositions that can more effectively degrade oral biofilm.

### SUMMARY OF THE INVENTION

The present invention provides enzymatic oral care compositions comprising a DNase, mutanase, and beta-glucanase. These enzymes act by degrading the extracellular DNA and exopolysaccharide components of oral biofilm, thereby improving oral health. This combination of enzymatic activities is highly active in prevention and removal of oral biofilm. In addition, the individual enzyme components are highly stable in the presence of wide range of oral care ingredients, making them very suitable for use in oral care formulations.

In a first aspect, the present invention relates to an enzymatic oral care composition comprising a DNase, a mutanase, a beta-glucanase, and at least one oral care ingredient.

In a second aspect, the present invention relates to a composition according to the first aspect for use as a medicament.

In a third aspect, the present invention relates to a composition according to the first aspect for use in the treatment of oral disease.

In a fourth aspect, the present invention relates to a kit of parts comprising:
a) a composition according to the first aspect; and
b) instructions for use.

### Definitions

Beta-glucanase: The term “beta-glucanase” means a polypeptide having endo-1,3(4)-β-glucanase activity (E.C. 3.2.1.6) activity that catalyzes the hydrolytic cleavage of (1,3)- and/or (1,4)-linkages in beta-D-glucans, thereby degrading beta-glucan. The term “beta-glucanase” and the expression “a polypeptide having endo-1,3(4)-β-glucanase activity” are used interchangeably throughout this application. For purposes of the present invention, endo-1,3(4)-β-glucanase activity may be determined according to the beta-glucanase activity assay described in the Examples section below.

Denture: The term “denture” is meant to cover dentures as such as well as braces, aligners, retainers, and the like.

DNase: The term “DNase” means a polypeptide having DNase (deoxyribonuclease) activity (E.C. 3.1.21 and E.C. 3.1.22) that catalyzes the hydrolytic cleavage of phosphodiester linkages in a DNA backbone, thus degrading DNA. The term “DNases” and the expression “a polypeptide having DNase activity” are used interchangeably throughout this application. For purposes of the present invention, DNase activity may be determined according to DNase activity assay I or DNase activity assay II described in the Examples section below.

Mutanase: The term “mutanase” means a polypeptide having endo-1,3-α-glucanase activity (E.C. 3.2.1.59) that catalyzes the hydrolytic cleavage of the 1,3-α-glycosidic linkages found in, e.g., mutan. The terms “mutanase” and “1,3-α-glucanase” and the expression “a polypeptide having endo-1,3-α-glucanase activity” are used interchangeably throughout this application. For purposes of the present invention, endo-1,3-α-glucanase activity may be determined according to the mutanase activity assay described in the Examples section below.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

### BRIEF DESCRIPTION OF THE FIGURES

FIG. 1 shows an example of the thermal stability data generated using the nanoDSF instrument. Panel A is an example of the data obtained (the ratio of the fluorescence emission at 350 nm to 330 nm) in triplicate for SEQ ID NO:2 as a function of temperature. Panel B shows the first derivative of the raw data in Panel A. The peak maximum in the first derivative plot corresponds to the mid-point of the thermal unfolding transition, referred to as Tm. In this example, Tm corresponds to 53.6° C. at pH 6.0 and is highly reproducible within the three replicates.

FIG. 2 shows the relative change in OPI scores from baseline to follow-up. The vertical bars denote the standard error.

FIG. 3 shows a boxplot showing the pairwise Bray-Curtis dissimilarity between baseline and follow-up sample for the placebo (white) and test (grey) group, with no statistical difference between groups (p=0.704).

FIG. 4 shows a bar plot showing the change in richness in saliva from baseline to follow up for each subject. We measured the richness by counting number of unique OTUs in each sample and the subjects are visualized according to placebo (white) and test (grey) group. No statistically significant change was found between the groups, but there was a trend towards an increased richness in the placebo group compared to the test group from baseline to follow-up (p=0.06). P numbers indicate individual subjects.

FIG. 5 shows a bar plot showing the change in richness in plaque from baseline to follow up for each subject. We measured the richness by counting number of unique OTUs in each sample and the subjects are visualized according to placebo (white) and test (grey) group. A higher increase in microbial richness between baseline and follow up was observed for the placebo group compared to the treatment group (p=0.04). P numbers indicate individual subjects.

FIG. 6 shows a bar plot showing the change in richness in saliva from baseline to follow up for each subject. We measured the richness by counting number of unique OTUs in each sample and the subjects are visualized according to placebo (white) and test (grey) group. No statistically significant change was found between the groups, but there was a trend towards an increased richness in the placebo group compared to the test group from baseline to follow-up (p=0.12). P numbers indicate individual subjects.

### SEQUENCE OVERVIEW

SEQ ID NO:1 is a DNase from Bacillus cibi.
SEQ ID NO:2 is a mutanase from Trichoderma harzianum.
SEQ ID NO:3 is a beta-glucanase from Aspergillus aculeatus.
SEQ ID NO:4 is a mutanase from Humicola insolens.
SEQ ID NO:5 is a mutanase from Trichoderma reesei.
SEQ ID NO:6 is a mutanase from Talaromyces bacillisporus.
SEQ ID NO:7 is a mutanase from Talaromyces apiculatus.
SEQ ID NO:8 is a mutanase from Streptomyces yanii.
SEQ ID NO:9 is a mutanase from Streptomyces alni.
SEQ ID NO:10 is a mutanase from Oribacterium sinus.
SEQ ID NO: 11 is a mutanase from Streptomyces galbus.
SEQ ID NO:12 is a mutanase from Streptomyces glomeratus.
SEQ ID NO:13 is a mutanase from Tetrasphaera sp-0185.
SEQ ID NO:14 is a mutanase from Kitasatospora phosalacinea.
SEQ ID NO:15 is a beta-glucanase from Aspergillus aculeatus.
SEQ ID NO:16 is a beta-glucanase from Aspergillus aculeatus.

### DETAILED DESCRIPTION OF THE INVENTION

The present invention provides enzymatic oral care compositions comprising a DNase, a mutanase, and a beta-glucanase. These enzymes act by degrading the extracellular DNA and exopolysaccharide components of oral biofilm, thereby improving oral health. This combination of enzymatic activities is highly active in prevention and removal of oral biofilm. In particular, clinical trials have shown that this enzyme combination is able to alter dental plaque morphology and reduce the amount of dental plaque without adversely affecting the oral microbiome. In addition, the individual enzyme components are highly stable in the presence of a wide range of oral care ingredients, making them very suitable for use in oral care formulations.

Thus, in a first aspect, the present invention relates to an enzymatic oral care composition comprising a DNase, a mutanase, a beta-glucanase, and at least one oral care ingredient.

### DNases

DNases degrade the DNA components of oral biofilm, in particular extracellular DNA. Any suitable DNase may be used in the oral care compositions of the invention. A DNase may cleave only double-stranded DNA or may cleave double stranded and single stranded DNA. The DNase is preferably an endo-deoxyribonuclease that cleaves or cut within the DNA backbone, but the DNase may also be an exo-deoxyribonuclease that cut or cleaves residues at the end of the DNA backbone.

Any suitable DNase may be used in enzymatic oral care compositions of the invention. Preferably, the DNase is obtained from a microorganism and the DNase is a microbial enzyme. The DNase is preferably of fungal or bacterial origin.

The DNase may be selected from any of the E.C. 3.1.21 enzyme classes, e.g., such as E.C. 3.1.21.X, where X=1, 2, 3, 4, 5, 6, 7, 8, 9, or 10, e.g., deoxyribonuclease I (E.C. 3.1.21.1), deoxyribonuclease IV (E.C. 3.1.21.2), type I site-specific deoxyribonuclease (E.C. 3.1.21.3), type II site-specific deoxyribonuclease (E.C. 3.1.21.4), type III site-specific deoxyribonuclease (E.C. 3.1.21.5), CC-preferring endo-deoxyribonuclease (E.C. 3.1.21.6), deoxyribonuclease V (E.C. 3.1.21.7), T(4) deoxyribonuclease II (E.C. 3.1.21.8), or T(4) deoxyribonuclease IV (E.C. 3.1.21.9), or crossover junction endo-deoxyribonuclease (E.C. 3.1.21.10).

The DNase may also be selected from any of the E.C. 3.1.22 enzyme classes, e.g., such as E.C. 3.1.22.Y, where Y=1, 2, or 5, e.g., deoxyribonuclease II (E.C. 3.1.22.1), Aspergillus deoxyribonuclease K(1) (E.C. 3.1.22.2), or deoxyribonuclease X (E.C. 3.1.22.5).

The DNase may be obtainable from Bacillus, e.g., Bacillus licheniformis, Bacillus subtilis, Bacillus sp-62451, Bacillus horikoshii, Bacillus sp-62451, Bacillus sp-16840, Bacillus sp-62668, Bacillus sp-13395, Bacillus horneckiae, Bacillus sp-11238, Bacillus cibi, Bacillus idriensis, Bacillus sp-62520, Bacillus sp-16840, Bacillus sp-62668, Bacillus algicola, Bacillus vietnamensis, Bacillus hwajinpoensis, Bacillus indicus, Bacillus marisflavi, Bacillus luciferensis, and Bacillus sp. SA2-6.

The DNase may also be obtained from any of the following organisms: Pyrenochaetopsis sp., Vibrissea flavovirens, Setosphaeria rostrate, Endophragmiella valdina, Corynespora cassiicola, Paraphoma sp. XZ1965, Monilinia fructicola, Curvularia lunata, Penicillium reticulisporum, Penicillium quercetorum, Setophaeosphaeria sp., Alternaria, Alternaria sp. XZ2545, Trichoderma reesei, Chaetomium thermophilum, Scytalidium thermophilum, Metapochonia suchlasporia, Daldinia fissa, Acremonium sp. XZ2007, Acremonium sp. XZ2414, Acremonium dichromosporum, Sarocladium sp. XZ2014, Metarhizium sp. HNA15-2, Isaria tenuipes Scytalidium circinatum, Metarhizium lepidiotae, Thermobispora bispora, Sporormia fimetaria, Pycnidiophora cf. dispera, Enviromental sample D, Enviromental sample O, Clavicipitaceae sp-70249, Westerdykella sp. AS85-2, Humicolopsis cephalosporioides, Neosartorya massa, Roussoella intermedia, Pleosporales, Phaeosphaeria or Didymosphaeria futilis.

Suitable DNases include those described, e.g., in WO 2017/060475, WO 2014/087011, WO 2015/155350, and WO 2015/155351.

In are preferred embodiment, the DNase exhibits improved stability in oral care formulations, e.g., toothpastes, mouthwashes, mints, lozenges, gums, etc., and/or in the presence of oral care components, e.g., sodium dodecyl sulphate (SDS) or fluoride sources such as sodium fluoride, sodium monofluorophosphate, calcium fluoride, or stannous fluoride. Examples of such DNases with improved stability are disclosed in WO 2020/099491.

In one embodiment, the oral care composition comprises a polypeptide having DNase activity obtained from Bacillus, e.g., obtainable from Bacillus cibi. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to SEQ ID NO:1, and has DNase activity. In a preferred embodiment, the polypeptide differs by up to 10 amino acids, e.g., 1, 2, 3, 4, 5, 6, 7, 8, 9, or 10, from the polypeptide shown in SEQ ID NO:1. In a preferred embodiment, the polypeptide comprises, consists essentially of, or consists of SEQ ID NO:1.

### Mutanases

Mutanases (EC 3.2.1.59, also known as 1,3-α-glucanases) are polypeptides having endo-1,3-α-glucanase activity that cleaves 1,3-α-glycosidic linkages found in various polysaccharides, e.g., mutan, which is a polysaccharide often found in oral biofilm. Mutanases are particularly useful against biofilm generated by the cariogenic microorganism Streptococcus mutans.

Any suitable mutanase may be used in enzymatic oral care compositions of the invention. Preferably, the mutanase is obtained from a microorganism and the mutanase is a microbial enzyme. The mutanase is preferably of fungal or bacterial origin.

Suitable mutanases have been derived from Trichoderma (Hasegawa et al., (1969), Journal of Biological Chemistry 244, p. 5460-5470; Guggenheim and Haller, (1972), Journal of Dental Research 51, p. 394-402) and from strains of Streptomyces (Takehara et al., (1981), Journal of Bacteriology 145, p. 729-735), Cladosporium resinae (Hare et al. (1978), Carbohydrate Research 66, p. 245-264), Pseudomonas sp. (U.S. Pat. No. 4,438,093), Flavobacterium sp. (JP 77038113), Bacillus circulans (JP 63301788) and Aspergillus sp.

A particularly suitable mutanase may be produced by a strain of the genus Trichoderma, in particular from a strain of Trichoderma harzianum, such as Trichoderma harzianum CBS 243.71. Other suitable mutanases may be produced by a strain of the genus Penicillium, in particular a strain of Penicillium funiculosum, such as Penicillium funiculosum NRRL 1768, or a strain of Penicillium lilacinum, in particular Penicillium lilacinum NRRL 896, or a strain of Penicillium purpurogenum, such as the strain of Penicillium purpurogenum CBS 238.95, or a strain of the genus Pseudomonas, or a strain of Flavobacterium sp., or a strain of Bacillus circulans or a strain of Aspergillus sp., or a strain of Streptomyces sp.

In one embodiment, the oral care composition comprises a polypeptide having endo-1,3-α-glucanase activity obtainable from Trichoderma, e.g., obtainable from Trichoderma harzianum. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:2, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:2. In a preferred embodiment, the polypeptide having endo-1,3-α-glucanase activity comprises, consists essentially of, or consists of SEQ ID NO:2.

In one embodiment, the oral care composition comprises a polypeptide having endo-1,3-α-glucanase activity obtainable from Humicola, e.g., obtainable from Humicola insolens. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:4, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:4. In a preferred embodiment, the polypeptide having endo-1,3-α-glucanase activity comprises, consists essentially of, or consists of SEQ ID NO:4.

In one embodiment, the oral care composition comprises a polypeptide having endo-1,3-α-glucanase activity obtainable from Trichoderma, e.g., obtainable from Trichoderma reesei. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:5, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:5. In a preferred embodiment, the polypeptide having endo-1,3-α-glucanase activity comprises, consists essentially of, or consists of SEQ ID NO:5.

In one embodiment, the oral care composition comprises a polypeptide having endo-1,3-α-glucanase activity obtainable from Talaromyces, e.g., obtainable from Talaromyces bacillisporus. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:6, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:6. In a preferred embodiment, the polypeptide having endo-1,3-α-glucanase activity comprises, consists essentially of, or consists of SEQ ID NO:6.

In one embodiment, the oral care composition comprises a polypeptide having endo-1,3-α-glucanase activity obtainable from Talaromyces, e.g., obtainable from Talaromyces apiculatus. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:7, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:7. In a preferred embodiment, the polypeptide having endo-1,3-α-glucanase activity comprises, consists essentially of, or consists of SEQ ID NO:7.

In one embodiment, the oral care composition comprises a polypeptide having endo-1,3-α-glucanase activity obtainable from Streptomyces, e.g., obtainable from Streptomyces yanii. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:8, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:8. In a preferred embodiment, the polypeptide having endo-1,3-α-glucanase activity comprises, consists essentially of, or consists of SEQ ID NO:8.

In one embodiment, the oral care composition comprises a polypeptide having endo-1,3-α-glucanase activity obtainable from Streptomyces, e.g., obtainable from Streptomyces alni. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:9, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:9. In a preferred embodiment, the polypeptide having endo-1,3-α-glucanase activity comprises, consists essentially of, or consists of SEQ ID NO:9.

In one embodiment, the oral care composition comprises a polypeptide having endo-1,3-α-glucanase activity obtainable from Oribacterium, e.g., obtainable from Oribacterium sinus. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:10, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:10. In a preferred embodiment, the polypeptide having endo-1,3-α-glucanase activity comprises, consists essentially of, or consists of SEQ ID NO:10.

In one embodiment, the oral care composition comprises a polypeptide having endo-1,3-α-glucanase activity obtainable from Streptomyces, e.g., obtainable from Streptomyces galbus. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:11, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:11. In a preferred embodiment, the polypeptide having endo-1,3-α-glucanase activity comprises, consists essentially of, or consists of SEQ ID NO:11.

In one embodiment, the oral care composition comprises a polypeptide having endo-1,3-α-glucanase activity obtainable from Streptomyces, e.g., obtainable from Streptomyces glomeratus. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:12, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:12. In a preferred embodiment, the polypeptide having endo-1,3-α-glucanase activity comprises, consists essentially of, or consists of SEQ ID NO:12.

In one embodiment, the oral care composition comprises a polypeptide having endo-1,3-α-glucanase activity obtainable from Tetrasphaera, e.g., obtainable from Tetrasphaera sp-0185. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:13, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:13. In a preferred embodiment, the polypeptide having endo-1,3-α-glucanase activity comprises, consists essentially of, or consists of SEQ ID NO:13.

In one embodiment, the oral care composition comprises a polypeptide having endo-1,3-α-glucanase activity obtainable from Kitasatospora, e.g., obtainable from Kitasatospora phosalacinea.

In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:14, and has endo-1,3-α-glucanase activity. In one aspect, the polypeptides differ by up to 10 amino acids, e.g., 1, 2, 3, 4, 5, 6, 7, 8, 9, or 10, from the mature polypeptide shown in SEQ ID NO:14. In a preferred embodiment, the polypeptide having endo-1,3-α-glucanase activity comprises, consists essentially of, or consists of SEQ ID NO:14.

### Beta-Glucanases

Beta-glucanases (EC 3.2.1.6) are polypeptides having endo-1,3(4)-β-glucanase activity that that catalyzes the hydrolytic cleavage of (1,3)- and/or (1,4)-linkages in beta-D-glucans.

Any suitable beta-glucanase may be used in enzymatic oral care compositions of the invention. Preferably, the beta-glucanase is obtained from a microorganism and the beta-glucanase is a microbial enzyme. The beta-glucanase is preferably of fungal or bacterial origin.

In one embodiment, the oral care composition comprises a polypeptide having endo-1,3(4)-β-glucanase activity obtained from Aspergillus, e.g., obtainable from Aspergillus aculeatus. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to SEQ ID NO:3, and has endo-1,3(4)-β-glucanase activity. In a preferred embodiment, the polypeptide differs by up to 10 amino acids, e.g., 1, 2, 3, 4, 5, 6, 7, 8, 9, or 10, from the polypeptide shown in SEQ ID NO:3. In a preferred embodiment, the polypeptide comprises, consists essentially of, or consists of SEQ ID NO:3.

In one embodiment, the oral care composition comprises a polypeptide having endo-1,3(4)-β-glucanase activity obtained from Aspergillus, e.g., obtainable from Aspergillus aculeatus. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to SEQ ID NO:15, and has endo-1,3(4)-β-glucanase activity. In a preferred embodiment, the polypeptide differs by up to 10 amino acids, e.g., 1, 2, 3, 4, 5, 6, 7, 8, 9, or 10, from the polypeptide shown in SEQ ID NO:15. In a preferred embodiment, the polypeptide comprises, consists essentially of, or consists of SEQ ID NO:15. In a preferred embodiment, the polypeptide comprises, consists essentially of, or consists of amino acid residues 1 to 741 of SEQ ID NO:15.

In one embodiment, the oral care composition comprises a polypeptide having endo-1,3(4)-β-glucanase activity obtained from Aspergillus, e.g., obtainable from Aspergillus aculeatus. In a preferred embodiment, the polypeptide has a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to SEQ ID NO:16, and has endo-1,3(4)-β-glucanase activity. In a preferred embodiment, the polypeptide differs by up to 10 amino acids, e.g., 1, 2, 3, 4, 5, 6, 7, 8, 9, or 10, from the polypeptide shown in SEQ ID NO:16. In a preferred embodiment, the polypeptide comprises, consists essentially of, or consists of SEQ ID NO:16. In a preferred embodiment, the polypeptide comprises, consists essentially of, or consists of amino acid residues 6 to 396 of SEQ ID NO:16. In a preferred embodiment, the polypeptide comprises, consists essentially of, or consists of amino acid residues 1 to 312 of SEQ ID NO:16. In a preferred embodiment, the polypeptide comprises, consists essentially of, or consists of amino acid residues 6 to 312 of SEQ ID NO:16.

### Stability in Oral Care Formulations

The enzymes (i.e., DNase, mutanase, and beta-glucanase) included in oral care compositions of the invention are highly stabile in formulations and/or formats suitable for oral care, in particular formulations or formats such as toothpastes, mouthwashes, lozenges, mints, gums, candy, etc. The high stability, e.g., on par or improved stability, may be on par or improved physical and/or chemical stability. On par or improved chemical stability, i.e., on par or improved stability in the presence of another agent (e.g., another enzyme, an active ingredient, an excipient, or a solvent) may occur when these enzymes and the other agent are co-formulated and/or co-administered, preferably upon co-formulation.

In the context of the present invention, the term “on par chemical stability” means that the chemical stability of an enzyme in the presence of (or, alternatively stated, co-formulated with) a particular oral care ingredient or component is within +/−5% of the chemical stability of the same enzyme alone (i.e., in the absence of said oral care ingredient).

In the context of the present invention, the term “improved chemical stability” means that the chemical stability of an enzyme in the presence of (or, alternatively stated, co-formulated with) a particular oral care ingredient or component is improved more than 5%, e.g., 10%, 15%, 20%, 25%, 30%, 40%, 50%, 60%, 70%, 80%, 90%, 100%, or even more, compared to the chemical stability of the same enzyme alone (i.e., in the absence of said oral care ingredient).

For purposes of the present invention, chemical stability may be determined according to Example 1 below as thermal stability defined by the thermal unfolding transition midpoint (Tm) in the presence of a particular oral care ingredient.

In one embodiment, at least one, e.g., at least two, or all, of DNase, mutanase, and beta-glucanase has on par or improved chemical stability in the presence of at least one oral care ingredient selected from the group consisting of benzoate (preferably sodium benzoate), EDTA, ethanol, glycerol, phosphate (preferably sodium phosphate or potassium phosphate), sorbitol, potassium sorbate, fluoride (preferably sodium fluoride, sodium monofluorophosphate, calcium fluoride, or stannous fluoride), hydrogen peroxide, and mannitol.

In one embodiment, at least one, e.g., at least two, or all, of DNase, mutanase, and beta-glucanase has on par or improved chemical stability in the presence of at least one oral care ingredient selected from the group consisting of benzoate (e.g., sodium benzoate), EDTA, ethanol, glycerol, (e.g., sodium phosphate), sorbitol, potassium sorbate, fluoride (e.g., sodium fluoride), hydrogen peroxide, and mannitol.

In one embodiment, the oral care composition comprises benzoate, e.g., sodium benzoate, and at least one, e.g., at least two, or all, of DNase, mutanase, and beta-glucanase has on par or improved chemical stability in the presence of benzoate, e.g., sodium benzoate. Preferably, the at least one, e.g., at least two, or all, of DNase, mutanase, and beta-glucanase has on par or improved chemical stability in the presence of 0.01-5% benzoate, more preferably 0.05-2.5% benzoate, even more preferably 0.1-1% benzoate, most preferably 0.1-0.5% benzoate. Preferably, the at least one, e.g., at least two, or all, of DNase, mutanase, and beta-glucanase has on par or improved chemical stability in the presence of 1-100 mM benzoate, more preferably 5-50 mM benzoate, most preferably 10-35 mM benzoate.

In one embodiment, the oral care composition comprises EDTA, and at least one, e.g., at least two, or all, of DNase, mutanase, and beta-glucanase has on par or improved chemical stability in the presence of EDTA. Preferably, the at least one, e.g., at least two, or all, of DNase, mutanase, and beta-glucanase has on par or improved chemical stability in the presence 0.1-10 mM EDTA, more preferably 0.5-5 mM EDTA, most preferably 1 mM EDTA.

In one embodiment, the oral care composition comprises ethanol, and at least one, e.g., at least two, or all, of DNase, mutanase, and beta-glucanase has on par or improved chemical stability in the presence of ethanol. Preferably, the at least one, e.g., at least two, or all, of DNase, mutanase, and beta-glucanase has on par or improved chemical stability in the presence of 0.1-20% ethanol, more preferably 1-10% ethanol, even more preferably 2.5-7.5% ethanol, most preferably 5% ethanol. Preferably, the at least one, e.g., at least two, or all, of DNase, mutanase, and beta-glucanase has on par or improved chemical stability in the presence of 1-100000 mM ethanol, more preferably 100-10000 mM ethanol, most preferably 1000 mM ethanol.

In one embodiment, the oral care composition comprises glycerol, and at least one, e.g., at least two, or all, of DNase, mutanase, and beta-glucanase has on par or improved chemical stability in the presence of glycerol. Preferably, the at least one, e.g., at least two, or all, of DNase, mutanase, and beta-glucanase has on par or improved chemical stability in the presence of 1-50% glycerol, more preferably 5-40% glycerol, most preferably 10-30% glycerol. Preferably, the at least one, e.g., at least two, or all, of DNase, mutanase, and beta-glucanase has on par or improved chemical stability in the presence of 100-10000 mM glycerol, more preferably 500-5000 mM glycerol, even more preferably 750-4000 mM glycerol, most preferably 1000-3250 mM glycerol.

In one embodiment, the oral care composition comprises phosphate, e.g., sodium phosphate or potassium phosphate, and at least one, e.g., at least two, or all, of DNase, mutanase, and beta-glucanase has on par or improved chemical stability in the presence of phosphate, e.g., sodium phosphate or potassium phosphate. Preferably, the at least one, e.g., at least two, or all, of DNase, mutanase, and beta-glucanase has on par or improved chemical stability in the presence of 1-50 mM phosphate, more preferably 2.5-25 mM phosphate, even more preferably 5-10 mM phosphate.

In one embodiment, the oral care composition comprises sorbitol, and at least one, e.g., at least two, or all, of DNase, mutanase, and beta-glucanase has on par or improved chemical stability in the presence of sorbitol. Preferably, the at least one, e.g., at least two, or all, of DNase, mutanase, and beta-glucanase has on par or improved chemical stability in the presence of 0.1-70% sorbitol, more preferably 1-60% sorbitol, even more preferably 5-50% sorbitol, most preferably 10-40% sorbitol. Preferably, the at least one, e.g., at least two, or all, of DNase, mutanase, and beta-glucanase has on par or improved chemical stability in the presence of 100-10000 mM sorbitol, more preferably 250-5000 mM sorbitol, even more preferably 500-2500 mM sorbitol, most preferably 550-2200 mM sorbitol.

In one embodiment, the oral care composition comprises sorbate, e.g., sodium sorbate, potassium sorbate, or calcium sorbate, and at least one, e.g., at least two, or all, of DNase, mutanase, and beta-glucanase has on par or improved chemical stability in the presence of sorbate, e.g., sodium sorbate, potassium sorbate, or calcium sorbate. Preferably, the at least one, e.g., at least two, or all, of DNase, mutanase, and beta-glucanase has on par or improved chemical stability in the presence of 0.01-5% sorbate, more preferably 0.05-2.5% sorbate, even more preferably 0.1-1% sorbate, most preferably 0.1-0.5% sorbate. Preferably, the at least one, e.g., at least two, or all, of DNase, mutanase, and beta-glucanase has on par or improved chemical stability in the presence of 1-100 mM sorbate, more preferably 5-75 mM sorbate, even more preferably 7.5-50 mM sorbate, most preferably 10-35 mM sorbate.

In one embodiment, the oral care composition comprises fluoride, e.g., sodium fluoride, sodium monofluorophosphate, calcium fluoride, or stannous fluoride, and at least one, e.g., at least two, or all, of DNase, mutanase, and beta-glucanase has on par or improved chemical stability in the presence of fluoride, e.g., sodium fluoride, sodium monofluorophosphate, calcium fluoride, or stannous fluoride. Preferably, the at least one, e.g., at least two, or all, of DNase, mutanase, and beta-glucanase has on par or improved chemical stability in the presence of 1-5000 ppm fluoride, more preferably 500-2500 ppm fluoride, most preferably 1,000-1500 ppm fluoride. Preferably, the at least one, e.g., at least two, or all, of DNase, mutanase, and beta-glucanase has on par or improved chemical stability in the presence of 1-100 mM fluoride, more preferably 5-75 mM fluoride, even more preferably 10-50 mM fluoride, most preferably 20-40 mM fluoride.

In one embodiment, the oral care composition comprises peroxide, e.g., hydrogen peroxide, and at least one, e.g., at least two, or all, of DNase, mutanase, and beta-glucanase has on par or improved chemical stability in the presence of peroxide, e.g., hydrogen peroxide. Preferably, the at least one, e.g., at least two, or all, of DNase, mutanase, and beta-glucanase has on par or improved chemical stability in the presence of 1-1000 mM peroxide, more preferably 50-750 mM peroxide, most preferably 100-500 mM peroxide.

In one embodiment, the oral care composition comprises mannitol, and at least one, e.g., at least two, or all, of DNase, mutanase, and beta-glucanase has on par or improved chemical stability in the presence of mannitol. Preferably, the at least one, e.g., at least two, or all, of DNase, mutanase, and beta-glucanase has on par or improved chemical stability in the presence of 1-1000 mM mannitol, more preferably 150-750 mM mannitol, most preferably 250-550 mM mannitol.

### Oral Care Compositions, Ingredients and Formats

The enzymatic oral care compositions of the invention comprise DNase, a mutanase, a beta-glucanase, and at least one oral care ingredient.

In one embodiment, the DNase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:1; preferably the DNase comprises, consists essentially of, or consists of SEQ ID NO:1.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:2; preferably the mutanase comprises, consists essentially of, or consists of SEQ ID NO:2.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:4; preferably the mutanase comprises, consists essentially of, or consists of SEQ ID NO:4.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:5; preferably the mutanase comprises, consists essentially of, or consists of SEQ ID NO:5.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:6; preferably the mutanase comprises, consists essentially of, or consists of SEQ ID NO:6.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:7; preferably the mutanase comprises, consists essentially of, or consists of SEQ ID NO:7.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:8; preferably the mutanase comprises, consists essentially of, or consists of SEQ ID NO:8.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:9; preferably the mutanase comprises, consists essentially of, or consists of SEQ ID NO:9.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:10; preferably the mutanase comprises, consists essentially of, or consists of SEQ ID NO:10.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:11; preferably the mutanase comprises, consists essentially of, or consists of SEQ ID NO:11.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:12; preferably the mutanase comprises, consists essentially of, or consists of SEQ ID NO:12.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:13; preferably the mutanase comprises, consists essentially of, or consists of SEQ ID NO:13.

In one embodiment, the mutanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:14; preferably the mutanase comprises, consists essentially of, or consists of SEQ ID NO:14.

In one embodiment, the beta-glucanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:3; preferably the beta-glucanase comprises, consists essentially of, or consists of SEQ ID NO:3.

In one embodiment, the beta-glucanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:15; preferably the beta-glucanase comprises, consists essentially of, or consists of SEQ ID NO:15.

In one embodiment, the beta-glucanase has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:16; preferably the beta-glucanase comprises, consists essentially of, or consists of SEQ ID NO:16.

In a preferred embodiment, the oral care composition comprises:
a) a DNase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:1;
b) a mutanase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:2; and
c) a beta-glucanase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity compared to SEQ ID NO:3.

In a preferred embodiment, the oral care composition comprises:
a) a DNase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:1;
b) a mutanase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:2; and
c) a beta-glucanase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity compared to SEQ ID NO:15 OR compared to the polypeptide defined by amino acid residues 1 to 741 of SEQ ID NO:15.

In a preferred embodiment, the oral care composition comprises:
a) a DNase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:1;
b) a mutanase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:2; and
c) a beta-glucanase having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity compared to SEQ ID NO:16 OR compared to the polypeptide defined by amino acid residues 6 to 396 of SEQ ID NO:16 OR compared to the polypeptide defined by amino acid residues 1 to 312 of SEQ ID NO:16 OR compared to the polypeptide defined by amino acid residues 6 to 312 of SEQ ID NO:16.

In a preferred embodiment, the oral care composition comprises:
a) a DNase comprising, consisting essentially of, or consisting of SEQ ID NO:1;
b) a mutanase comprising, consisting essentially of, or consisting of SEQ ID NO:2; and
c) a beta-glucanase comprising, consisting essentially of, or consisting of SEQ ID NO:3 OR a beta-glucanase comprising, consisting essentially of, or consisting of SEQ ID NO:15 OR a beta-glucanase comprising, consisting essentially of, or consisting of SEQ ID NO:16.

In a preferred embodiment, the oral care composition comprises:
a) a DNase comprising, consisting essentially of, or consisting of SEQ ID NO:1;
b) a mutanase comprising, consisting essentially of, or consisting of SEQ ID NO:2; and
c) a beta-glucanase comprising, consisting essentially of, or consisting of SEQ ID NO:3 and a beta-glucanase comprising, consisting essentially of, or consisting of SEQ ID NO:15 and beta-glucanase comprising, consisting essentially of, or consisting of SEQ ID NO:16.

In a preferred embodiment, the oral care composition comprises:
a) a DNase comprising, consisting essentially of, or consisting of SEQ ID NO:1;
b) a mutanase comprising, consisting essentially of, or consisting of SEQ ID NO:2; and
c) a beta-glucanase comprising, consisting essentially of, or consisting of SEQ ID NO:3.

In a preferred embodiment, the oral care composition comprises:
a) a DNase comprising, consisting essentially of, or consisting of SEQ ID NO:1;
b) a mutanase comprising, consisting essentially of, or consisting of SEQ ID NO:2; and
c) a beta-glucanase comprising, consisting essentially of, or consisting of SEQ ID NO:15.

In a preferred embodiment, the oral care composition comprises:
a) a DNase comprising, consisting essentially of, or consisting of SEQ ID NO:1;
b) a mutanase comprising, consisting essentially of, or consisting of SEQ ID NO:2; and
c) a beta-glucanase comprising, consisting essentially of, or consisting of SEQ ID NO:16.

The at least one oral care ingredient may be varied according to the type of oral care composition as well as the desired characteristics and/or activities of the oral care compositions. For the purpose of the present invention, the terms “ingredient” and “component” are used interchangeably in relation to oral care compositions.

An oral care composition of the invention may be an internal oral care composition such as toothpaste or toothpaste tablet, dental cream, mouthwash or mouthwash tablet, mouth rinse, lozenges, pastilles, chewing gum, confectionary, candy, and the like, which is designed to remove biofilm inside the oral cavity, e.g., biofilm residing on teeth, on soft tissues of the oral cavity, and on dentures residing in the oral cavity.

An oral care composition of the invention may also be an external oral care composition such as denture cleaning solution, denture cleaning tablet, denture cleaning powder, and the like, which is designed to remove biofilm from dentures that have been removed from the oral cavity for cleaning.

In a preferred embodiment, the oral care composition is an internal oral care composition, and the at least one oral care component is selected from the group consisting of abrasives, humectants, solvents, thickening agents, binding agents, buffering agents, foaming agents, foaming modulators, sweetening agents, softening agents, plasticizing agents, flavoring agents, coloring agents, therapeutic agents, anti-microbial agents, tartar-controlling agents, fluoride sources, preservatives, detergents, surfactants, coloring agents, buffering agents, softeners, plasticizers, whitening agents, bleaching agents, gum-base ingredients, and bulking agents.

Although the oral care ingredients mentioned herein are categorized by a general header according to a functionality, this is not to be construed as a limitation, as an ingredient may comprise additional functionalities as will be appreciated by the skilled person.

### Toothpaste, Dental Cream, Mouthwash, and Mouth Rinse

Internal oral care compositions of the invention in the form of toothpaste or toothpaste tablet, dental cream, mouthwash or mouthwash tablet, and mouth rinse may include oral care ingredients selected from the following categories:

| | | Amount |
|---|---|---|
| Type of ingredient | Examples | (weight %) |
| Abrasives | Calcium carbonate, sodium bicarbonate, | 0 to 70% |
| | diatomite, charcoal, dicalcium phosphate | |
| | dehydrate, hydrated silica, hydrated alumina | |
| Humectants | Glycerol, sorbitol and propylene glycol | 0 to 80% |
| Solvents | Water, ethanol, water/ethanol mixtures | 0.001 to 95% |
| Thickening/binding | Xanthan gum, cellulose gum (sodium | 0.01 to 20% |
| agents | carboxymethyl cellulose), hydroxyethyl | |
| | cellulose, carrageenan, glycerin, potassium | |
| | thiocyanate, PVM/MA copolymer, carbomer, | |
| | arrowroot powder | |
| Buffering agents | Sodium hydroxide, sodium phosphate, di- and | 0.001 to 10% |
| | trisodium phosphate, sodium citrate, calcium | |
| | glycerophosphate, | |
| Formulation and | Sodium lauryl sulfate (SLS), sodium lauryl | 0.001 to 10% |
| foaming agents | sarcosinate, sodium lauroyl glutamate, sodium | |
| (detergents, | methyl cocoyl taurate, Steareth-30, coco- | |
| surfactants) | glucoside, propylene glycol (1,2-propanediol), | |
| | Poloxamer 407, Polysorbate 20, Polysorbate | |
| | 80 | |
| Foaming | PEGs (polyethylene glycols) | 0.001 to 10% |
| agents/modulators | | |
| Sweetening agents | Xylitol, sorbitol, erythritol, mannitol, sodium | 0.001 to 60% |
| | saccharin, maltitol, stevia, sucralose, | |
| | acesulfame K, aspartame, hydrogenated | |
| | starch hydrolysate | |
| Softener/plasticizer | Lecithin, hydrogenated vegetable oils, glycerol | 0.001 to 5% |
| | ester, lanolin, methyl ester, pentaerythritol | |
| | ester, stearic acid, sodium and potassium | |
| | stearates | |
| Flavoring agents | Peppermint oil, spearmint oil | 0.001 to 5% |
| Coloring agents | Titanium dioxide, beta-carotene, | 0.001 to 20% |
| | FD&C Blue 1 Aluminum Lake, FD&C Blue 2 | |
| | Lake, FD&C Yellow 5 Aluminum Lake, FD&C | |
| | Yellow 5 | |
| Preservatives | Sodium benzoate, methyl paraben, ethyl | 0.001 to 30% |
| | paraben, citric acid, potassium sorbate, | |
| | calcium citrate, propylene glycol | |
| Whitening/bleaching | Hydrogen peroxide, sodium hypochlorite, | 0.01 to 10% |
| agents | sodium perborate | |
| Therapeutic agents | calcium fluoride, sodium fluoride, triclosan, | 0.001 to 50% |
| (including anti- | various essential oils (thymol, menthol), | |
| bacterial, anti- | benzoic acid, sodium benzoate, zinc gluconate, | |
| carries, anti- | aloe vera, chloride, cetylpyridinuim chloride, | |
| inflammatory | domiphen bromide, methyl salicylate, | |
| agents) | chlorhexidine digluconate | |

### Toothpaste

Toothpastes and dental creams/gels typically include abrasives, solvents, humectants, detergents/surfactants, thickening and binding agents, buffering agents, flavoring agents, sweetening agents, fluoride sources, therapeutic agents, enzymes, coloring agents, and preservatives.

In a preferred embodiment, the present invention relates to enzymatic oral care compositions in the form of a toothpaste or dental cream comprising a DNase, a mutanase, a beta-glucanase, and at least one oral care ingredient, wherein the at least one oral care ingredient is selected from the following ingredients:

| | | Amount |
|---|---|---|
| Type of ingredient | Examples | (weight %) |
| Abrasive | Calcium carbonate, sodium | 10 to 50% |
| | bicarbonate, hydrated silica, hydrated | |
| | Alumina | |
| Solvent | Water | 5 to 40% |
| Humectant | Glycerin and sorbitol | 20 to 70% |
| Detergent/Surfactant | Sodium lauryl sulphate (SLS), sodium | 1 to 3% |
| | lauryl sarcosinate, | |
| | cocamidopopybetaine | |
| | and steareth-30 | |
| Thickening and | Natural gums (e.g. xanthan gum, | 1 to 2% |
| binding agent | cellulose gum) or synthetic polymers | |
| | (e.g., PVM/MA copolymer, carbomer). | |
| | hydroxyethyl cellulose, carrageenan, | |
| | glycerin. | |
| Buffering agent | Sodium citrate, calcium | 1 to 60% |
| | glycerophosphate, sodium hydroxide, | |
| | di- and trisodium pyrophosphate, | |
| | disodium phosphate | |
| Flavoring agent | Peppermint oil, spearmint oil | 0 to 2% |
| Sweetening agent | Xylitol, sorbitol, erythritol, saccharin, | 0 to 2% |
| | stevia, sucralose | |
| Fluoride source | Sodium fluoride, calcium fluoride, | 0.001 to 10% |
| | stannous fluoride, sodium | |
| | monofluorophosphate | |
| Therapeutic agent | Triclosan, zinc gluconate, aloe vera | 0.001 to 50% |
| | extracts, chloride, cetylpyridinuim | |
| | chloride, domiphen bromide, methyl | |
| | salicylate, chlorhexidine, chlorhexidine | |
| | digluconate | |
| Coloring agent | Titanium dioxide | 0 to 0.5% |
| Preservative | Sodium gluconate, citric acid, sodium | 0.05 to 0.5% |
| | benzoate | |

An oral care composition of the invention may be a toothpaste comprising the following ingredients (in weight % of the final toothpaste composition):
Abrasive: 10 to 70%
Humectant: 0 to 80%
Thickening agent: 0.1 to 20%
Binding agent: 0.01 to 10%
Sweetening agent: 0.1 to 5%
Foaming agent: 0 to 15%
Enzymes (DNA, mutanase, and beta-glucanase): 0.01 to 10%

### Mouthwash

Mouthwashes and mouth rinses of the invention, including plaque removing liquids, typically comprise enzymes, a carrier liquid, detergents/surfactants, buffering agents, flavoring agents, humectants, sweetening agents, therapeutic agents, fluoride sources, coloring agents, preservatives, and enzymes.

In a preferred embodiment, the present invention relates to enzymatic oral care compositions in the form of a mouthwash or mouth rinse comprising a DNase, a mutanase, a beta-glucanase, and at least one oral care ingredient, wherein the at least one oral care ingredient is selected from the following ingredients:

| Type of ingredient | Example |
|---|---|
| Carrier liquid, | Water, ethanol (18-27%), water/ethanol |
| usually 60-99% | mixture |
| Detergent/surfactant | Poloxamer 407, Polysorbate 20, Polysorbate |
| | 80, Steareth-30 |
| Buffering agent | Sodium Hydroxide, (Di)-Sodium phosphate, |
| | Sodium citrate, Benzoic acid, Sodium |
| | Benzoate |
| Flavoring agent | Peppermint oil, spearmint oil |
| Humectants | Glycerin, Sorbitol |
| Sweetening agents | Saccharin, Xylitol |
| Therapeutic agent | Triclosan, zinc gluconate, aloe vera extracts, |
| | chloride, cetylpyridinuim chloride, domiphen |
| | bromide, methyl salicylate, chlorhexidine, |
| | chlorhexidine digluconate |
| Fluoride source | Sodium fluoride, calcium fluoride, stannous |
| | fluoride, or sodium monofluorophosphate |
| Coloring agent | Titanium oxide |
| Preservative | Sodium Benzoate, Benzoic acid, Citric acid, |
| | Propylene glycol |

An oral care composition of the invention may be a mouthwash comprising the following ingredients (in weight % of the final mouthwash composition):
Water: 0 to 70%
Ethanol: 0 to 20%
Humectant: 0 to 20%
Surfactant: 0 to 2%
Enzymes (DNA, mutanase, and beta-glucanase): 0.01 to 10%
Other ingredients: 0 to 2% (e.g., flavors, sweeteners, fluoride sources).

The mouthwash composition may be buffered with an appropriate buffer, e.g., sodium citrate or phosphate in the pH range 6-7.5.

Relevant oral care components suitable for toothpastes, dental creams, mouthwashes, and mouth rinses is further detailed below. The skilled person is capable of varying the oral care components according to the type of oral care composition as well as the desired characteristics and/or activities of the specific oral care composition. An oral care composition may not necessarily comprise all of the mentioned ingredients.

### Abrasives

Abrasive polishing material might be incorporated into the oral care composition of the invention. According to the invention said abrasive polishing material includes alumina and hydrates thereof, such as alpha alumina trihydrate, magnesium trisilicate, magnesium carbonate, kaolin, aluminosilicates, such as calcined aluminum silicate and aluminum silicate, calcium carbonate, zirconium silicate, bentonite, silicium dioxide, sodium bicarbonate, and also powdered plastics, such as polyvinyl chloride, polyamides, polymethyl methacrylate, polystyrene, phenol-formaldehyde resins, melamine-formaldehyde resins, urea-formaldehyde resins, epoxy resins, powdered polyethylene, silica xerogels, hydrogels and aerogels, and the like.

Also suitable as abrasive agents are calcium pyrophosphate, water-insoluble alkali metaphosphates, poly-metaphosphates, dicalcium phosphate and/or its dihydrate, dicalcium orthophosphate, tricalcium phosphate, particulate hydroxyapatite, and the like. It is also possible to employ mixtures of these substances.

Silica dental abrasives of various types are preferred because of their unique benefits of exceptional dental cleaning and polishing performance without unduly abrading tooth enamel or dentine, and which have a good compatibility with other possible ingredients, like metal ions and fluoride.

Dependent on the oral care composition, the abrasive product may be present in from 0 to 70% by weight, preferably from 1% to 70%.

For toothpastes the abrasive material content typically lies in the range of from 10% to 70% by weight of the final tooth-paste product.

### Humectants

Humectants are employed to prevent loss of water from, e.g., toothpastes and to avoid hardening of toothpastes upon exposure to air. Some humectants also give a desirable sweetness of flavor to toothpaste and mouthwash compositions. Suitable humectants for use in oral care compositions according to the invention include the following compounds and mixtures thereof: glycerol, polyol, sorbitol, xylitol, maltitol, lactitol, polyoxyethylene, polyethylene glycols (PEG), polypropylene glycols, propylene glycol, 1,3-propanediol, 1,4-butanediol, hydrogenated partially hydrolyzed polysaccharides and the like, coconut fatty acid, amide of N-methyl-taurine, and Pluronic®.

Humectants are in generally present in from 0% to 80%, preferably 5 to 70% by weight.

### Thickening/Binding Agents

Suitable thickening and/or binding agents include silica, starch, tragacanth gum, xanthan gum, karaya gum, carrageenans (extracts of Irish moss), gum arabic, alginates, pectin, cellulose derivatives, such as hydroxyethyl cellulose, sodium carboxymethyl cellulose, hydroxypropyl cellulose and hydroxyethyl propyl cellulose, polyacrylic acid and its salts, polyvinylpyrrolidone and carboxyvinyl polymers, as well as inorganic thickeners such as amorphous silica compounds. These agents stabilize the oral care compositions of the invention.

Thickeners may be present in toothpaste, dental creams and gels as well as in mouthwashes in an amount of from 0.1 to 20% by weight, and binders to the extent of from 0.01 to 10% by weight of the final product.

### Foaming Agents and Foaming Modulators

As foaming agent soap, anionic, cationic, non-ionic, amphoteric and/or zwitterionic surfactants can be used, either alone or in combinations. These may be present at levels of from 0% to 15%, preferably from 0.1% to 13%, more preferably from 0.25% to 10% by weight of the final product. Surfactants are only suitable to the extent that they do not exert an inactivation effect on the enzymes and other components included in the oral care composition. Useful surface-active agents include anionic, nonionic, and ampholytic compounds, with anionic compounds being preferred.

Examples of suitable surfactants include salts of the higher alkyl sulfates, such as sodium lauryl sulfate or other suitable alkyl sulphates having 8 to 18 carbon atoms in the alkyl group; sodium lauryl sulfoacetate, salts of sulfonated monoglycerides of higher fatty acids, such as sodium coconut monoglyceride sulfonate or other suitable sulfonated monoglycerides of fatty acids of 10 to 18 carbon atoms; salts of amides of higher fatty acid, e.g., 12 to 16 carbon atom acids, with lower aliphatic amino acids, such as sodium-N-methyl-N-palmitoyl tauride, sodium N-lauroyl-, N-myristoyl- and N-palmitoyl sarcosinates; salts of the esters of such fatty acids with isotopic acid or with glycerol monosulphate; such as the sodium salt of monosulphated monoglyceride of hydrogenated coconut oil fatty acids; salts of olefin sulfonates, e.g., alkene sulfonates or alkene sulfonates or mixtures thereof having 12 to 16 carbon atoms in the carbon chain of the molecule; and soaps of higher fatty acids, such as those of 12 to 18 carbon atoms, e.g., coconut fatty acids.

The cation of the salt may be sodium, potassium or mono-, di or triethanol amine. The nonionic surfactants include sucrose/fatty acid esters, maltose/fatty acid esters, maltitol/fatty acid esters, maltotriitol/fatty acid esters, maltotetraitol/fatty acid esters, maltopentaitol/fatty acid esters, maltohexaitol/fatty acid esters, mahoheptaitol/fatty acid esters, sorbitan/fatty acid esters, lactose/fatty acid esters, lactinose/fatty acid esters, polyoxyethylene/polyoxypropylene copolymers, polyoxyethylene alkyl ethers, polyoxyethylene/fatty acid esters, fatty acid alkanolamides, polyoxyethylene sorbitan/fatty acid esters, polyoxyethylene/hydrogenated castor oil, and polyglycerin/fatty acid esters.

Most preferred are sodium lauryl sulphate, sodium dodecylbenzene sulphonate and sodium lauryl sarcosinate.

Preferred foaming modulators include polyethylene glycols.

Foaming agents and foaming modulators may be present from in an amount of from 0% to 15% by weight, preferably from 0.01% to 10% by weight.

### Sweetening Agents

Suitable sweeteners include, but are not limited to, saccharin and water-soluble salts thereof, dextrose, sucrose, lactose, maltose, levulose, aspartame, cyclamate salts, D-tryptophan, dihydrochalchones, acesulphame, stevioside, levaudioside, glycyrrhizins, pellartine, thaumatin, p-methoxycinnamic aldehyde, hydrogenated starch hydrolysates, xylitol, sorbitol, erythritol, mannitol, and mixtures thereof.

Sweeteners may be present from in an amount of from 0.001% to 60% by weight, preferably from 0.01% to 50% by weight.

### Flavoring Agents

Flavoring agents are usually present in low amounts, such as from 0.01% to about 5% by weight, especially from 0.1% to 5%. The flavors that may be used in the invention include, but are not limited to, wintergreen oil, peppermint oil, spearmint oil, clove bud oil, menthol, anethole, methyl salicylate, eucalyptol, cassia, 1-inenthyl acetate, sage, eugenol, parsley oil, oxanone, alpha-irisone, marjoram, lemon, orange, cranberry, propenyl guaethol, cinnamon, vanillin, ethyl vanillin, heliotropine, 4-cis-heptenal, diacetyl, methylpara-tert-butyl phenyl acetate, carvone, cineole, menthone, cinnamic aldehyde, limonene, ocimene, n-decyl alcohol, citronellol, alpha-terpineol, methyl acetate, citronellyl acetate, methyl eugenol, linalool, thymol, rosemary oil, pimento oil, diatomaceous oil, eucalyptus oil, and mixtures thereof.

Coolants may also be part of the flavor system or added separately to the composition. Preferred coolants in the present compositions are the paramenthan carboxyamide agents such as N-ethyl-p-menthan-3-carboxamide (known commercially as ‘WS-3”), menthol, 3-1-menthoxypropanc-1,2-diol (“TK-10”), menthone glycerol acetal (“MGA”), menthyl lactate and mixtures thereof.

### Whitening/Bleaching Agents

Whitening/bleaching agents include H₂O₂ and may be added in amounts less than 5%, preferably from 0.05 to 4%, calculated on the basis of the weight of the final composition.

Other bleaching components which might be comprised by the present invention include, peroxydiphosphate, urea, peroxide, metal peroxides such as calcium peroxide, sodium peroxide, stronthium peroxide, magnesium peroxide, hypochlorite salts such as sodium hypochlorite, and the salts of perborate, persilicate, perphosphate and percarbonate such as sodium perborate, potassium persilicate and sodium percarbonate. The peroxide compounds can be stabilized by addition of a triphenylmethane dye, a chelating agent or antioxidants such as butylated hydroxy anisole (BHA) or butylated hydroxy toluene (BHT).

### Solvent

A solvent is usually added to compositions of the invention in an amount sufficient for giving the compositions a flowable form in case the compositions is; e.g., a toothpaste, dental cream or gel, or to dissolve the other components of a compositions, in case of, e.g., a mouthwash or mouth rinse.

Suitable solvents include water, ethanol and water/ethanol mixtures, which may be present in an amount of from 0.1% to 70%.

### Anti-Microbial Agents

The present invention also includes water-soluble anti-microbial agents, such as chlorhexidine, triclosan, digluconate, hexetidine, alexidine, quaternary ammonium antibacterial compounds, and water-soluble sources of certain metal ions such as zinc, copper, silver and stannous (e.g., zinc, copper and stannous chloride, and silver nitrate) may also be included.

Sparingly soluble zinc salts such as zinc citrate, zinc C14-alkyl maleate, zinc benzoate, zinc caproate, zinc carbonate might also be included used in the compositions of the present invention to prolong the anti-microbial effectiveness of zinc ions due to the slow dissolution of these zinc salts in saliva.

Anti-microbial agents may be present in an amount of from 0% to 50% by weight, preferably from 0.01% to 40% by weight, most preferably from 0.1% to 30% by weight.

### Tartar-Controlling Agent

Compositions of the invention may comprise a tartar-controlling agent such as inorganic phosphorous tartar-controlling agents including any of the pyrophosphates such as disodium pyrophosphate, dipotassium pyrophosphate, tetrapotassium pyrophosphate, tetrasodium pyrophosphate, and mixtures thereof.

Organic phosphorous compounds that may serve as tartar-controlling agents include polyphosphonates such as disodium ethane-1-hydroxy-1, 1-diphosphonate (EHDP), methanediphosphonic acid, and 2-phosphonobutane-1 2,4-tricarboxylic acid.

Tartar-controlling agents may be present in an amount of from 0% to 10% by weight, preferably from 0.1% to 5% by weight.

### Preservatives

Suitable preservatives include sodium benzoate, potassium sorbate, p-hydroxybenzoate esters, methyl paraben, ethyl paraben, propyl paraben, citric acid, calcium citrate, and mixtures thereof.

Preservatives may be present in an amount of from 0% to 40% by weight, preferably from 0.01% to 30% by weight.

### Fluoride Sources

Compositions of the invention may also comprise ingredients that can be used as fluoride source. Preferred soluble fluoride sources include sodium fluoride, potassium fluoride, stannous fluoride, indium fluoride, sodium monofluorophosphate, sodium hexafluorosilicate, zinc fluoride, lithium fluoride, aluminum fluoride, acidulated phosphate fluoride, ammonium bifluoride, titanium tetrafluoride, and amine fluoride.

Especially preferred are sodium fluoride and sodium monofluorophosphate.

Fluoride sources may be present in an amount of from 0% to 20% by weight, preferably from 0.01% to 15% by weight, most preferably from 0.1% to 10% by weight.

In a preferred embodiment, the at least one oral care ingredient is a fluoride source; preferably the fluoride source is selected from the group consisting of sodium fluoride, calcium fluoride, stannous fluoride, or sodium monofluorophosphate

### Coloring Agents

Coloring agents or pigments suitable for oral care compositions of the invention include non-toxic, water-insoluble inorganic pigments such as titanium dioxide and chromium oxide greens, ultramarine blues and pinks and ferric oxides as well as water insoluble dye lakes prepared by extending calcium or aluminum salts of FD&C dyes on alumina such as FD&C Green No. 1 lake, FD&C Blue No. 2 lake, FD&C Red No. 30 lake, FD&C Yellow No. 16 lake, and FD&C Yellow No. 10.

A preferred opacifier is titanium dioxide.

Coloring agents may be present in an amount of from 0% to 20% by weight, preferably from 0.01% to 15% by weight, most preferably from 0.1% to 10% by weight.

### Buffering Agents

The oral care compositions of present invention may also include buffering agents, i.e., pH-adjusting agents, such as alkali metal hydroxides, carbonates, sesquicarbonates, borates, silicates, phosphates, imidazole, and mixtures thereof.

Specific buffering agents include monosodium phosphate, trisodium phosphate, sodium hydroxide, potassium hydroxide, alkali metal carbonate salts, sodium carbonate, imidazole, pyrophosphate salts, sodium citrate, hydrochloric acid, sodium hydroxide, triethanolamine, triethylamine, lactic acid, malic acid, fumaric acid, tartaric acid, phosphoric acid and mixtures of these.

Buffering agents may be present in an amount of from 0% to 10% by weight, preferably from 0.01% to 5% by weight.

### Chewing Gum

When the oral composition according to the invention is a chewing gum, it can be any known type of chewing gum, such as chewing gum pieces optionally coated, as well as sticks or chewing gum provided with an arbitrary desired shape in response to the intended use. The chewing gum preparation can be of any quality including the bubble gum quality.

In a preferred embodiment, the present invention relates to enzymatic oral care compositions in the form of a chewing gum comprising a DNase, a mutanase, a beta-glucanase, and at least one oral care ingredient, wherein the at least one oral care ingredient is selected from elastomer, softening agent, plasticizing agent, emulsifier, wax, coloring agent, sweetening agent, flavoring agent, bulking agent, and thickening agent.

### Gum Base Ingredients

Chewing gum is traditionally considered as being comprised of a water-insoluble or base portion and a water-soluble portion that contains flavoring agents, sweetening agents, and coloring agents. The gum base part of the gum is a masticatory substance which imparts the chew characteristics to the final product. It defines the release profile of flavors and the sweeteners and plays a significant role in the gum product. The flavors, sweeteners and colors can be thought of as providing the sensory appeal aspects of the chewing gum. No limitations as to the chewing gum bases used in a chewing gum preparation according to the invention exist. Conventional chewing gum bases available for instance from Dansk Tyggegummi Fabrik A/S, L. A. Dreyfus or Cafasa Gum SIA, are usually suitable, but specially made formulations can also be used. The formulation depends on the desired type of chewing gum or the desired type of structure. Suitable raw materials for gum bases include the substances according to the U.S. Chewing Gum Base Regulations—Code of Federal Regulations, Title 21, Section 172,615 and in accordance with other national and international lists (or positive lists) and include elastomers, resins, waxes, polyvinyl acetates, oils, fats, emulsifiers, fillers and antioxidants.

The gum base usually comprises from 15 to 90% by weight, preferably from 30 to 40% by weight, more preferably from 5 to 25% of the final product.

Elastomers provide the chew, springiness or bounce to the base and control bubble and flavor release in the final chewing gum. They may be any water-insoluble polymer known in the art. They include styrene butadiene copolymers (SBR) and non-SBR types, both natural and synthetic.

Examples of natural elastomers include, without limitation, rubbers such as rubber latex (natural rubber) and guayule, and gums such as chicle, jelutong, balata, guttapercha, lechi capsi, sorva, crown gum, nispero, rosidinha, perillo, niger gutta, tunu, gutta kay, pendare, leche de vaca, chiquibul, crown gum, and the like, and mixtures thereof. Examples of synthetic elastomers include, without limitation, polyisobutylene, isobutylene-isoprene copolymers (butylrubber), polyethylene, polybutadiene, styrenebutadiene copolymers, polyisoprene, and the like, and mixtures thereof.

The amounts of elastomer (rubbers) employed in the gum base composition will vary greatly depending upon various factors such as the type of gum base used (adhesive, or conventional, bubble or standard) the consistency of the gum base composition desired, and the other components used in the composition to make the final chewing gum product. In general, the elastomer is present in the gum base composition in an amount of from about 15% to about 60%, preferably from about 25% to about 30%, by weight based on the total weight of the gum base composition.

Elastomer solvents aid in softening or plasticizing the elastomer component. In doing so they provide a bulkiness to the chew.

Elastomer solvents include, but are not limited to, natural rosin esters and synthetic derivatives of, e.g., terpenes. Examples of elastomer solvents suitable for use herein include tall oil rosin ester; partially hydrogenated wood and gum rosin; the glycerol esters of wood and gum rosin, partially hydrogenated wood/gum rosin, partially dimerized wood and gum rosin, polymerized wood and gum rosin, and tall oil rosin; the deodorized glycerol ester of wood rosin; the pentaerythritol esters of wood and gum rosin; partially hydrogenated wood and gum rosin; the methyl ester of partially hydrogenated wood rosin; methyl, glycerol and pentaerythritol esters of rosins and modified rosins such as hydrogenated, dimerized and polymerized rosins; terpene resins such as polymers of alpha-pinene or beta-pinene, terpene hydrocarbon resins; polyterpene; and the like, and mixtures thereof. The elastomer solvent may be employed in the gum base composition in an amount of from about 2% to about 40%, and preferably from about 7% to about 15% by weight of the gum base composition.

Polyvinyl acetates provide stretch or elasticity to the gum base. They also affect chew bulkiness, softness and bubble, hydrophilic character and flavor release.

The amounts of the different molecular weight polyvinyl acetates present in the gum base composition should be effective to provide the finished chewing gum with the desired chew properties, such as integrity, softness, chew bulkiness, film-forming characteristic, hydrophilic character, and flavor release. The total amount of polyvinyl acetate used in the gum base composition is usually from about 45% to about 92% by weight based on the total gum base composition. The vinyl polymers may possess a molecular weight ranging from about 2000 Da up to about 95,000 Da.

Typically, the low molecular weight polyvinyl acetate has a weight average molecular weight of from about 2,000 Da to about 14,000 Da. The medium molecular weight polyvinyl acetate typically has a weight average molecular weight of from about 15,000 Da to 55,000 Da. The high molecular weight polyvinyl acetate typically has a weight average molecular weight of from 55,000 Da to about 95,000 Da but may range as high as 500,000 Da.

Waxes, fats, and oils plasticize the elastomer mixture and improve the elasticity of the gum base. Waxes can provide a soft or firm chew, affect the flavor release and provide bulkiness and smoothness to the gum base. Fats and oils provide a soft chew. The fats, oils and waxes may be use individually or in combination or the gum base may be a wax free gum base.

Waxes when used, may be of mineral, animal vegetable or synthetic origin. Non-limiting examples of mineral waxes include petroleum waxes such as paraffin and microcrystalline waxes, animal waxes include beeswax, vegetable waxes include carnauba, candellila, rice bran, esparto, flax and sugarcane, and synthetic waxes include those produced by the Fischer-Tropsch synthesis, and mixtures thereof.

Suitable oils and fats usable in gum compositions include hydrogenated or partially hydrogenated vegetable or animal fats, such as cottonseed oil, soybean oil, coconut oil, palm kernel oil, beef tallow, hydrogenated tallow, lard, cocoa butter, lanolin and the like; fatty acids such as palmitic, oleic, stearic, linoleic, lauric, myristic, caproic, caprylic, decanoic or esters and salts as sodium stearate and potassium stearate. These ingredients when used are generally present in amounts up to about 7% by weight of the gum composition, and preferably up to about 3.5% by weight of the gum composition.

Preferred as softeners are the hydrogenated vegetable oils and include soybean oil and cottonseed oil which may be employed alone or in combination. These softeners provide the gum base composition with good texture and soft chew characteristics. These softeners are generally employed in an amount from about 5% to about 14% by weight of the gum base composition.

Emulsifiers aid in dispersing the immiscible components of the gum base composition into a single stable system. They provide hydrophilic character to a gum base and aid in plasticizing the resins and polyvinyl acetates. They also affect the softness of the base and the bubble character of the base. Typical emulsifiers include acetylated monoglyceride, glyceryl monostearate, lecithin, fatty acid monoglycerides, diglycerides, propylene glycol monostearate, lecithin, triacetin, glyceryl triacetate and the like, and mixtures thereof.

Preferred emulsifiers are glyceryl monostearate and acetylated monoglycerides. These serve as plasticizing agents. The emulsifiers may be employed in an amount of from about 2% to about 15% by weight of the gum base composition, and preferably from about 7% to about 11% by weight of the gum base composition.

The fats, oils, waxes, emulsifiers and certain sugar bulking agents are often grouped together and referred to as softening agents. Because of the low molecular weight of these ingredients, the softeners are able to penetrate the fundamental structure of the gum base making it plastic and less viscous. Useful plasticizers and softeners of the above include lanolin, palmitic acid, oleic acid, stearic acid, sodium stearate, potassium stearate, glyceryl triacetate, glyceryl lecithin, glyceryl monostearate, propylene glycol nonastearate, acetylated monoglyceride, glycerin, fully unsaturated vegetable oils such as nonhydrogenated cottonseed oil, hydrogenated vegetable oils, petroleum waxes, sorbitan monostearate, tallow, and the like, and mixtures thereof and also include high fructose corn syrup, corn syrup, sorbitol solution, hydrogenated starch hydrolysate, and the like, and mixtures thereof.

The amount of softener present should he an effective amount to provide a finished chewing gum with the desired chew bulkiness and softness. When used as softeners these materials are generally employed in the gum base composition in an amount of up to about 25%, and preferably in an amount of from about 1% to about 17%, by weight of the gum base composition.

The gum base may further contain a surfactant. Examples of suitable surfactants include polyoxyethylene (20) sorbitan monoleate, polyoxyethylene (20) sorbitan monolaurate, polyethylene (4) sorbitan monolaurate, polyoxyethylene (20) sorbitan monopalmitate, polyoxyethylene (20) sorbitan monostearate, polyoxyethylene, (4) sorbitan monostearate, polyoxyethylene (20) sorbitan tristearate, polyoxyethylene (5) sorbitan monooleate, polyoxyethylene (20) sorbitan trioleate, sorbitan monolaurate, and the like. The amount of surfactant present should be effective to provide the finished chewing gum with the desired softness. Typically, the surfactant is employed in the base in an amount of from about 0.5% to about 3.0% by weight based on the total weight of the gum base.

The gum base composition of this invention may also include effective amounts of fillers sometimes referred to as bulking agents. These materials add firmness and bulk and affect the texture and the flavor release of the chewing gum. Useful fillers include organic and inorganic compounds (mineral adjuvants) such as calcium carbonate, magnesium carbonate, ground limestone, magnesium silicate, calcium phosphate, cellulose polymers, clay, alumina, aluminum hydroxide, aluminum silicate, tale, tricalcium phosphate, dicalcium phosphate, and the like, and mixtures thereof.

These fillers or adjuvants may be used in the gum base compositions in various amounts. The amount of the filler present should be effective to provide a finished chewing gum with the desired flavor release and integrity. Typically, the filler is employed in the gum base composition in an amount from about 1% to about 40%, and preferably from about 5% to about 20%, by weight of the gum base composition.

The gum base may also comprise an antioxidant to provide improved stability, lessen any oil-taste and provide longer shelf life. Typical non-limiting examples of antioxidants are butylated hydroxytoluene (BHT), butylated hydroxy anisole (BHA), propyl gallate. Mixtures thereof may also be used.

### Other Gum Ingredients

The remaining ingredients in chewing gum compositions are conventional and usually comprise from 10 to 85% by weight of the final product.

Examples thereof are sweetening agents, softeners, coloring agents, bulking agents, thickening agents, and flavoring agents of the type and in the amounts conventionally used for chewing gum.

Suitable flavoring agents those flavors known to the skilled artisan such as natural and artificial flavors. These flavorings may be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, wintergreen oil (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Other useful flavorings are artificial, natural and synthetic fruit flavors such as vanilla, and citrus oils including lemon, orange, lime, grapefruit, and fruit essences including apple, pear, peach, grape, strawberry, raspberry, cherry, plum, pineapple, apricot and so forth. These flavoring agents may be used in liquid or solid form and may be used individually or in admixtures. Commonly used flavors include mints such as peppermint, menthol, artificial vanilla, cinnamon derivatives, and various fruit flavors, whether employed individually or in admixture.

Other useful flavoring agents include aldehydes and esters such as cinnamyl acetate, cinnamaldehyde, citrate diethylacetal, dihydrocarvyl acetate, eugenyl formate, p-methyl anisole, and so forth may be used. Generally, any flavoring or food additive may be used.

Further examples of aldehyde flavorings include, but are not limited to, acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2-hexenal (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), cherry, grape, strawberry shortcake, mixtures thereof and the like.

The amount of flavoring agent employed herein is normally a matter of preference subject to such factors as the type of final chewing gum composition, the individual flavor, the gum employed, and the strength of flavor desired. Thus, the amount of flavoring may be varied in order to obtain the result desired in the final product and such variations are within the capabilities of those skilled in the art without the need for undue experimentation. In gum compositions, the flavoring agent is generally present in amounts from about 0.02% to about 5% by weight of the chewing gum composition.

The chewing gum compositions generally include bulking agents. These bulking agents (carders, extenders) may be water-soluble and include bulking agents selected from the group consisting of, but not limited to, monosaccharides, disaccharides, polysaccharides, sugar alcohols, and mixtures thereof; sorbitol, xylitol, maltitol, mannitol, isomalt (a racemic mixture of alpha-D-glucopyranosyl-1,6-mannitol and alpha-D-glucopyranosyl-1,6-sorbitol manufactured under the tradename Palatinit™ by Suddeutsche Zucker), glycerol, aspartame, Lycasin® glycerol, galactitol acesulphame K, saccharine and salts thereof, cyclamate and salts thereof, neohesperidine dihydrochalcone, glycyrrhizinic acid and salts thereof, thaumantine and sucralose as well as mixtures thereof or mixtures thereof with other suitable sweeteners, maltodextrins; hydrogenated starch hydrolysates; hydrogenated hexoses; hydrogenated disaccharides; minerals, such as calcium carbonate, talc, titanium dioxide, dicalcium phosphate, celluloses and the and the like, and mixtures thereof. Bulking agents may be used in amounts up to about 60%, and preferably in amounts from about 25% to about 60%, by weight of the chewing gum composition.

The chewing gum compositions may also include a high intensity sweetening agent (sweeteners). High intensity sweetening agents have a sweetness intensity substantially greater than that of sucrose. Examples of suitable intense sweeteners include:
a) water-soluble naturally-occurring intense sweeteners such as dihydrochalcones, monellin, steviosides, glycyrrhizin, dihydroflavenol, and L-aminodicarboxylic acid aminoalkonoic acid ester amides, such as those disclosed in in U.S. Pat. No. 4,619,834, and mixtures thereof;
b) water-soluble artificial sweeteners including the soluble saccharin salts such as sodium or calcium saccharin salts, cyclamate salts, the sodium, ammonium or calcium salts of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfam-K), the free acid form of saccharin, and the like, and mixtures thereof;
c) dipeptide based sweeteners including L-aspartic acid derived sweeteners such as 1-aspartyl-L-phenylalanine methyl ester (Aspartame) and materials described in U.S. Pat. No. 3,492,131, L-alpha-aspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine, L-aspartyl-L-(1-cyclohexen)-alanine, and the like, and mixtures thereof;
d) water-soluble intense, sweeteners derived from naturally-occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose®; examples of chlorodeoxysucrose and chlorodeoxygalactosucrose derivatives include but are not limited to: to 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-Ideoxy-beta-D-fructo-furanoside, or 4,1′-dichloro-4,1′-dideoxygalactosucrose; 1′,6′-dichloro-1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galactopyranosyl-1,6-dichloro-1,6-dideoxy-beta-D-fluctofuranoside, or 4,6,1′,6′-tetrachloro-4,6,1′,6′-tetradeoxygalacto-sucrose; and 4,6,1′,6′-tetradeoxy-sucrose, and mixtures thereof; and
e) protein based intense sweeteners such as Thaumaoccous daniclii (Thaumatin I and II). The amount of sweetener employed in the chewing gum composition will vary with the sweetener selected for a particular chewing gum. Thus, for any given sweetener a sufficient amount of sweetener is used to provide the level of sweetness desired. The saccharide sweeteners and sugar alcohols described above are usually used in an amount of from about 1% to about 70% and preferably in an amount of from about 40% to about 50%, by weight based on the total weight of the chewing gum composition. The intense sweeteners described above are usually used in an amount of up to about 1%, preferably from about 0.05% to about 0.4%, by weight based on the total weight of the chewing gum composition.

The coloring agents useful in the present invention are used in amounts effective to produce the desired color. These coloring agents include pigments, which may be incorporated in amounts up to about 6%, by weight of the gum composition. A preferred pigment, titanium dioxide, may be incorporated in amounts up to about 2%, and preferably less than about 1%, by weight of the gum composition. The colorants may also include natural food colors and dyes suitable for food, drug and cosmetic applications. These colorants are known as F.D.& C. dyes and lakes. The materials acceptable for the foregoing uses are preferably water-soluble. Illustrative non-limiting examples include the indigoid dye known as F.D.& C. Blue No. 2, which is the disodium salt of 5,5-indigotindisulfonic acid. Similarly, the dye known as F.D.& C. Green No. 1 comprises a triphenylmethane dye and is the monosodium salt of 4-[4-(N-ethyl-N-p-sulfoniumbenzylamino)diphenylmethylene]-[1-(N-ethyl-N-p-sulfoniumbenzyl)-delta-2,5-cyclo-hexadieneimine].

Examples of thickening agents include methyl cellulose, alginates, carrageenan, xanthan gum, gelatin, carob, tragacanth, and locust bean, emulsifiers, such as lecithin and glyceryl monostearate, acidulants such as malic acid, adipic acid, citric acid, tartaric acid, fumaric acid, and mixtures thereof.

The plasticizers, softening agents, emulsifiers, waxes, and antioxidants discussed above as being suitable for use in the gum base may also be used in the chewing gum composition.

### Active Gum Ingredients

Oral care compositions of the invention in the form of a chewing gum may also contain various active ingredients such as antimicrobial agents, Zn salts, fluorides, and urea.

Moreover, the oral composition according to the invention may, if desired, include any other active ingredients, such as anti-caries agents, anti-calculus agents, anti-plaque agents, anti-periodontal agents, anti-fungal agents, anti-smoking agents, anti-cold agents, agents against gingivitis, etc.

The antimicrobials used in the compositions can be any of a wide of cationic antimicrobial agents such as quaternary ammonium compounds (e.g., cetyl pyridinium chloride) and substituted guanidines such as chlorhexidine and the corresponding compound alexidine. Mixtures of cationic anti-microbials may also be used in the present invention.

Antimicrobial quaternary ammonium compounds include those in which one or two of the substituents on the quaternary nitrogen has a carbon chain length (typically alkyl group) of some 8 to 20, typically 10 to 18 carbon atoms while the remaining substituents (typically alkyl or benzyl group) have a lower number of carbon atoms, such as 1 to 7 carbon atoms, typically methyl or ethyl groups. Dodecyl trimethyl ammonium bromide, tetradecyl pyridinium chloride, tetradecyl ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis 2-ethyl-hexyl)-5-methyl hexa hydropyrimidine and benzethonium chloride are exemplary of typical quaternary ammonium antibacterial agents. Other compounds are the bis[4-(R-amino)-1-pyridinium]alkanes as disclosed in U.S. Pat. No. 4,206,215, Jun. 3, 1980, incorporated herein by reference. The pyridinium compounds are the preferred quaternary ammonium compounds.

The cationic antimicrobial is generally used in the present compositions at a level of from about 0.02% to about 1%, preferably from about 0.3% to about 0.7% most preferably from about 0.3% to about 0.5%.

As easily soluble zinc salt it is in principle possible to use any physiologically acceptable, easily soluble zinc salt of an inorganic or organic acid, said salt being able to release zinc ions and being approved for the intended use, such as in foodstuffs, cosmetics or pharmaceutical products. Non-limiting examples are for instance zinc citrate, zinc sulphate, zinc lactate, zinc chloride, zinc acetate as well as mixtures thereof. Among these salts zinc acetate is preferred.

The zinc salt used must be easily soluble such that a release is ensured in the oral cavity of an amount of zinc ions efficient for the purpose aimed at within a suitable period of time.

Advantageously, the zinc salt is present in the oral composition in an amount of from 0.001 to 1.25% by weight. The amount used depends on the administration form and the intended use and is adapted such that an amount of zinc ions efficient for the intended use is released.

As taste-masking salt is used at least one salt selected among sodium chloride, ammonium chloride and physiologically acceptable alkali metal, alkaline earth metal and/or ammonium carbonates.

The alkali metal is in particular sodium or potassium, whereas the alkaline earth metal advantageously is calcium or magnesium. Particularly preferred taste-masking salts are sodium, potassium and magnesium carbonates, sodium chloride, ammonium chloride as well as mixtures thereof.

The taste-masking salt is advantageously used in the oral composition in an amount of from 0.05 to 6.25% by weight, more preferred from 0.25 to 3.50% by weight, such as from 0.50 to 2.50% by weight.

The amount used of taste-masking salt for masking the taste of zinc can in each case be determined by a person skilled in the art and depends on the particular zinc salt in question and the selected administration form.

Urea is used as an anticariogenic product for neutralizing the acid produced in dental plaque subsequent to eating or drinking. Beyond urea the composition also can contain pharmacologically acceptable substances capable of releasing urea under the conditions prevailing in the mouth.

Examples thereof are salts and addition compounds between urea and inorganic compounds such as magnesium sulphate, calcium phosphate, sodium chloride, etc.

The urea content of the composition according to the invention varies between 0.05% by weight and 80% by weight, preferably between 0.2% by weight and 25% by weight.

The chewing gum compositions may be prepared using standard techniques and equipment known to those skilled in the art. The apparatus useful in accordance with the present invention comprises mixing and beating apparatus as well.

### Lozenges and Pastilles

Lozenges are flavored medicated dosage forms intended to be sucked and held in the mouth or pharynx. They may contain vitamins, antibiotics, antiseptics, local anesthetics, antihistamines, decongestants, corticosteroids, astringents, analgesics, aromatics, demulcents, or combinations of these ingredients. Lozenges may take various shapes, the most common being the flat, circular, octagonal, and biconvex forms. Another type, called bacilli, are in the form of short rods or cylinders. A soft variety of lozenge, called a pastille, consists of medicament in a gelatin or glycerogelatin base or in base of acacia, sucrose, and water (H. A. Lieberman, Pharmaceutical Dosage Forms: Tablets, Volume 1 (1980), Marcel Dekker, Inc., New York, N.Y.).

In a preferred embodiment, the present invention relates to enzymatic oral care compositions in the form of a lozenge or pastille comprising a DNase, a mutanase, a beta-glucanase, and at least one oral care ingredient, wherein the at least one oral care ingredient is selected from lubricant, bulking agent, sweetening agent, and flavoring agent.

### Lubricants

The use of a lubricant in the manufacture of compressed lozenges is to facilitate the release of the lozenge from the die in which it is formed. The lubricant used in the present invention is a solid material which is not charged, and which will not interfere (e.g., complex) with the cationic antimicrobial. The material should preferably be water insoluble. One type of suitable material meeting these requirements is a non-toxic hydrocarbon fat or derivative. Examples include hydrogenated tallow and hydrogenated vegetable oil. Polyethylene glycols may also be used as a lubricant so long as they are solid materials which generally means having a molecular weight in the 4000 Da to 6000 Da range. These materials can also be used as a filler as noted below.

Mixtures of lubricants may also be used in the present invention. The lubricant is used at level of from about 0.1% to about 4.0% preferably from about 0.5% to about 2%.

### Lozenge Vehicle

The term “lozenge vehicle” is used herein to denote the material(s) which carries the active ingredients, enzymes and therapeutic agents, as well as the lubricant. These materials are also known as bulking agents or fillers. Since the vehicle is non-cariogenic, the vehicle should be free of sucrose and similar materials.

Acceptable filler materials include mannitol, sorbitol, xylitol, polyethylene glycol and non-cariogenic dextrans. The fillers may be used alone or in combination.

Mannitol is a naturally occurring sugar alcohol and is available as a fine powder. It has a sweetness of only about 50% of that of sucrose. However, mannitol's negative heat of solution enables it to impart a pleasant, cooling sensation in the mouth as the lozenge dissolves.

Sorbitol is a chemical isomer of mannitol and possesses a similar degree of sweetness. Its heat of solution, being negative, also provides for a pleasant, cooling sensation in the mouth. Sorbitol is available either as free flowing granules or as a crystalline powder. Polyethylene glycols (PEG's) can also be used in the present compositions. These materials are polymers of ethylene oxide with the generalized formula HOCH₂ (CH₂OCH₂)ₙCH₂OH. The use of PEG's alone is not favored but their use in combination with other fillers is acceptable. The molecular weights found most desirable are between 4000 Da and 6000 Da.

Fillers are generally used in the present invention at a level of from about 85% to about 99.8%, preferably from about 90% to about 98%, most preferably from about 94% to about 97%.

### Other Lozenge Components

Acceptable lozenges may be manufactured using just an active ingredient, the lubricant and the filler material as outlined above. However, in order to make the lozenges more acceptable from an aesthetic viewpoint, generally included are materials such as spray-dried or encapsulated flavors or liquid flavors adsorbed onto a suitable diluent. Spray-dried or encapsulated flavors are preferred. Suitable flavors include oil of peppermint, oil of wintergreen, oil of sassafras, oil of spearmint and oil of clove. Sweetening agents are also acceptable for use in the present compositions. Suitable agents include aspartame, acesulfame, saccharin, dextrose and levulose. Sweetening and flavoring agents are generally used in the compositions of this invention at levels of from about 0.1% to about 2%, preferably from about 0.25% to about 1.5%.

It is also acceptable to have a solid form of a water-soluble fluoride compound present in the present lozenges in an amount sufficient to give a fluoride concentration of from about 0.0025% to about 5.0% by weight, preferably from about 0.005% to about 2.0% by weight, to provide additionally anticaries effectiveness. Preferred fluorides are sodium fluoride, stannous fluoride, indium fluoride and sodium monofluorophosphate. The lozenges may also contain various active ingredients such as anti-microbial agents, Zn salts, fluorides, and urea (supra).

### Confectionaries and Candy

In a preferred embodiment, the present invention relates to enzymatic oral care compositions in the form of a confectionary or candy comprising a DNase, a mutanase, a beta-glucanase, and at least one oral care ingredient, wherein the at least one oral care ingredient is selected from coloring agent, sweetening agent, flavoring agent, and oil-modifying agent.

The preparation of confectionery formulations is historically well known and has changed little through the years. Confectionery items have been classified as either “hard” confectionery or “soft” confectionery. The volatile oil-modifying agent of the present invention can be incorporated by admixing the modifying agent into conventional hard and soft confections.

Hard confectionery may be processed and formulated by conventional means. In general, a hard confectionery has a base composed of a mixture of sugar and other carbohydrate bulking agents kept in an amorphous or glassy condition. This form is considered a solid syrup of sugars generally having from about 0.5% to about 1.5% moisture. Such materials normally contain up to about 92% corn syrup, up to about 55% sugar and from about 0.1% to about 5% water, by weight of the final composition. The syrup component is generally prepared from corn syrups high in fructose but may include other materials. Further ingredients such as flavorings, sweeteners, acidulants, colorants and so forth may also be added.

Such confectionery may be routinely prepared by conventional methods such as those involving fire cookers, vacuum cookers, and scraped-surface cookers also referred to as high-speed atmospheric cookers.

Fire cookers involve the traditional method of making a candy base. In this method, the desired quantity of carbohydrate bulking agent is dissolved in water by heating the agent in a kettle until the bulking agent dissolves. Additional bulking agent may then be added, and cooking continued until a final temperature of 145 to 156° C. is achieved. The batch is then cooled and worked as a plastic-like mass to incorporate additives such as flavor, colorants and the like.

A high-speed atmospheric cooker uses a beat-exchanger surface, which involves spreading a film of candy on a heat exchange surface, the candy is heated to 165 to 170° C. in a few minutes. The candy is then rapidly cooled to 100 to 120° C. and worked as a plastic-like mass enabling incorporation of the additives, such as flavors, colorants and the like.

In vacuum cookers, the carbohydrate bulking agent is boiled to 125 to 132° C., vacuum is applied, and additional water is boiled off without extra heating. When cooking is complete, the mass is a semi-solid and has a plastic-like consistency. At this point, flavors, colorants, and other additives are admixed in the mass by routine mechanical mixing operations.

The optimum mixing required to uniformly mix the flavors, colorants and other additives during conventional manufacturing of hard confectionery is determined by the time needed to obtain a uniform distribution of the materials. Normally, mixing times of from 4 to 10 minutes have been found to be acceptable.

Once the candy mass has been properly tempered, it may be cut into workable portions or formed into desired shapes. A variety of forming techniques may be utilized depending upon the shape and size of the final product desired. A general discussion of the composition and preparation of hard confections may be found in H. A. Lieberman, Pharmaceutical Dosage Forms: Tablets, Volume 1 (1980), Marcel Dekker, Inc., New York, N.Y.

The apparatus useful in accordance with the present invention comprises cooking and mixing apparatus well known in the confectionery manufacturing arts, and election of the specific apparatus will be apparent to the artisan. In contrast, compressed tablet confections contain particular materials and are formed into structures under pressure.

These confections generally contain sugars in amounts up to about 95%, by weight of the composition, and typical tablet excipients such as binders and lubricants as well as flavoring agent, colorants and so forth. Similar to hard confectionery, soft confectionery may be utilized in this invention. The preparation of soft confections, such as nougat, involves conventional methods, such as the combination of two primary components, namely (1) a high boiling syrup such as corn syrup, hydrogenated starch hydrolysate or the like, and (2) a relatively light textured frappe, generally prepared from egg albumin, gelatin, vegetable proteins, such as soy derived compounds, sugarless milk derived compounds such as milk proteins, and mixtures thereof. The frappe is generally relatively light, and may, for example, range in density from about 0.5 to about 0.7 grams/cc.

The flavoring components of the confection are flavors having an associated bitter taste or other unpleasant after taste. These flavoring components may be chosen from natural and synthetic flavoring liquids such as volatile oils, synthetic flavor oils, flavoring aromatic and oils, liquids, oleoresins or extracts derived from plants, leaves, flowers, fruits, stew and combinations thereof. Non-limiting representative examples of volatile oils include spearmint oil, cinnamon oil, oil of wintergreen (methyl salicylate), peppermint oil, menthol, clove oil, bay oil, anise oil, eucalyptus oil, thyme oil, cedar leaf oil, oil of nutmeg, allspice oil, oil of sage, mace extract, oil of bitter almonds, and cassia oil. In addition, the confection may also contain artificial, natural or synthetic flavors including fruit flavors such as vanilla, and citrus oils including lemon, orange, grape, lime and grapefruit and fruit essences including apple, pear, peach, grape, strawberry, raspberry, cherry, plum, pineapple, apricot and so forth individual and mixed.

Other useful flavorings include aldehydes and esters such as benzaldehyde (cherry, almond), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyl-octanal (green fruit), and 2-dodecenal (citrus, mandarin), mixtures thereof and the like.

In the instance where sweeteners are utilized, the present invention contemplates the inclusion of those sweeteners well known in the art, including both natural and artificial sweeteners. The sweeteners may be chosen from the following non-limiting list: sugars such as sucrose, glucose (corn syrup), dextrose, invert sugar, fructose, and mixtures thereof, saccharin and its various salts such as the sodium or calcium salt; cyclamic acid and its various salts such as the sodium salt; the dipeptide sweeteners such as aspartame, dihydrachalcone compounds, glycyrrhizin; Stevia rebaudiana (Stevioside); chloro-derivatives of sucrose; dihydroflavinol; hydroxyguaiacol esters; L-amino dicarboxylic acid gem-diamines; L-aminodicarboxylic acid aminoalkenoic acid ester amides; and sugar alcohols such as sorbitol, sorbitol syrup, mannitol, xylitol, and the like. Also contemplated is the synthetic sweetener 3,6-dihydro-6-methyl-1,2,3-oxathiazin-4-one-2,2-dioxide, particularly the potassium (acesulfame-K), sodium and calcium salts thereof.

The confection may also include a colorant. The colorants may be selected from any of the numerous dyes suitable for food, drug and cosmetic applications, and known as FD&C dyes and the like. The materials acceptable for the foregoing spectrum of use are preferably water-soluble. Illustrative examples include indigoid dye, known as FD&C Blue No. 2, which is the disodium salt of 5,5′-indigotindisulfonic acid. Similarly, the dye known as FD&C Green No. 1 comprises a triphenylmethane dye and is the monosodium salts of 4-[4-N-ethyl-p-sulfobenzylamino)diphenylmethylane]-[1-(N-ethyl-N-p-sulfoniumbenzyl)-2-5-cyclohexadieneimine]. A full recitation of all FD&C and D&C dyes and their corresponding chemical structures may be found in the Kirk-Othmer Encyclopedia of Chemical Technology, in Volume 5.

The confectionary may also include a volatile oil-modifying agent such as Capsicum oleoresin. An oil-modifying agent is present in an amount, which is undetected as a separate ingredient in the oral cavity, but nevertheless has the ability to modify sensory perception of the volatile oil.

The oil-modifying agent is present in an amount of from about 1 to about 150 ppm of the confection. The Capsicum is available from Capsicum minimum, Capsicum frutescens, Capsicum annuum, and similar varieties. Commercially, the fruits of Capsicum are referred to as chilies or as peppers. These fruits are known for their extreme potency of bite, pungency and characteristic odor.

With respect to confectionery compressed tablet formulations, such will contain a tablet granulation base and various additives such as sweeteners and flavors. The tablet granulation base employed will vary depending upon factors such as the type of base used, friability desired and other components used to make the final product. These confections generally contain sugars in amounts up to 95% by weight of the composition.

The confectionery compressed tablet may additionally include tablet excipients such as binders or lubricants, as well as flavoring agents, coloring agents, and volatile oils and volatile oil-modifying agents.

The variations that one may practice with regard to these confections are wide ranging and within the ability of those skilled in the art particularly with regard to the use of additional composition fillers, flavoring agents, the use of coloring agents, etc.

### External Oral Care Compositions

An external oral care formulation, e.g., denture cleaning solution, denture cleaning tablet, denture cleaning powder, and the like, may include ingredients and/or substances selected from the following categories:

| Type of | | Amount |
|---|---|---|
| component | Examples | (weight %) |
| Carrier liquid | Water, ethanol (18-27%), | 0 to 95% |
| | water/ethanol mixture | |
| Disinfectant and | Sodium hypochlorite, Potassium | 0.001 to 95% |
| bleaching agents | Monopersulfate, Sodium Perborate | |
| Cleaning agents | Sodium bicarbonate, citric acid, | 0.001 to 95% |
| | sodium perborate, sodium | |
| | polyphosphate, potassium | |
| | monopersulfate | |
| Detergents | Sodium lauryl sulfate (SLS) | 0.001 to 10% |
| and surfactants | | |
| Foaming agents | Polyethylene glycols (PEGs) | 0.0001 to 10% |
| Preservatives | Sodium benzoate, citric acid, | 0.001 to 75% |
| Flavoring agents | Peppermint, spearmint | 0.001 to 5% |

In a preferred embodiment the at least on oral care ingredient is selected from the group consisting of carrier liquids, disinfectant and bleaching agents, cleaning agents, detergents and surfactants, foaming agents, preservatives, and flavoring agents.

### Other Oral Care Compositions

The enzymatic oral compositions of the invention may also be included in filaments suitable for use in dental cleaning, e.g., filaments useful as dental floss. Preferably, the oral care composition is coated onto the exterior of the filament. Thus, in a preferred aspect, the present invention relates to a filament comprising an enzymatic oral care composition comprising a DNase, a mutanase, a beta-glucanase, and at least one oral care ingredient, wherein the filament is suitable for dental cleaning.

### Treatment of Oral Disease

The enzymatic oral care compositions of the invention are suitable for use in the treatment of oral disease, wherein removal of biofilm is desired. The compositions of the invention are particularly suitable for treating periodontal diseases and dental caries.

Periodontal disease, also known as gum disease, is a set of inflammatory conditions caused by bacterial infection and subsequent biofilm build-up on the teeth and the tissues surrounding the teeth. Periodontal disease may be divided in terms of severity into the following categories: gingivitis (including plaque-induced gingivitis), chronic periodontitis, aggressive periodontitis, periodontitis as a manifestation of systemic disease, necrotizing ulcerative gingivitis/periodontitis, abscesses of the periodontium, and combined periodontic-endodontic lesions. Periodontal disease may further be considered either localized or generalized depending on the extent of the affected area.

Dental caries, also known as tooth decay or cavities, is caused by organic acids, such as lactic acid, being released by certain biofilm-forming bacteria residing in the oral cavity, including Streptococcus mutans and some Lactobacillus species. Dental caries may be associated with further complications such as inflammation of the tissue around the teeth, tooth loss, and infection or abscess formation. Dental caries may be classified by location, etiology, rate of progression, and affected hard tissues, for instance according to the G.V. Black classification (class I, II, III, IV, V, and VI).

In one aspect, the present invention relates to an enzymatic oral care composition comprising a DNase, a mutanase, a beta-glucanase, and at least one oral care ingredient for use as a medicament.

In one aspect, the present invention relates to an enzymatic oral care composition comprising a DNase, a mutanase, a beta-glucanase, and at least one oral care ingredient for use in the treatment of oral disease.

In a preferred embodiment, the present invention relates to an enzymatic oral care composition comprising a DNase, a mutanase, a beta-glucanase, and at least one oral care ingredient for use in the treatment of periodontal disease and/or dental caries.

In one aspect, the present invention relates to use of an enzymatic oral care composition comprising a DNase, a mutanase, a beta-glucanase, and at least one oral care ingredient for treatment or prophylactic treatment of a human subject.

In one aspect, the present invention relates to a method of treatment of a human subject, the method comprising administering an enzymatic oral care composition comprising a DNase, a mutanase, a beta-glucanase, and at least one oral care ingredient to a human subject.

In one aspect, the present invention relates to a method for removing oral biofilm, the method comprising contacting the biofilm with an oral care composition comprising a DNase, a mutanase, a beta-glucanase, and at least one oral care ingredient. In one embodiment, the enzymatic oral care composition is an external oral care composition, and the biofilm is located on an object; preferably the object is a denture. In one embodiment, the object is located inside or outside the oral cavity.

### Canine Oral Care Composition

The enzymatic oral care compositions of the invention may be canine oral care compositions. That is to say that the composition may be for use in a canine animal. Accordingly, an embodiment of the invention is directed to a composition for use as a canine oral care composition, wherein said composition comprises a DNase, a mutanase, and a beta-glucanase.

The composition may be formulated as toothpaste or toothpaste tablet, dental cream, or paste or cream for application to a consumable product. The consumable products may be in the form of a chew, a treat, dry food, or liquid supplement. The chew may be for example a dental stick, hard chew, a soft chew, a biscuit, a freeze-dried treat, and a jerky treat. Otherwise stated, in one embodiment, the composition is suitable for application to an oral cavity, preferably onto the teeth of canine and in an alternate embodiment the composition is suitable for application onto a canine consumable product, prior to consumption by the canine. Accordingly, as aspect of the invention is directed to an oral care composition of use as a toothpaste or dental cream or as a cream or paste.

In a further aspect, the composition is formulated as part of a consumable product. Accordingly, as further aspect of the invention is directed to a consumable product for canines comprising the composition of the invention.

As demonstrated in Example 4, composition of the invention is effective against pathogens found in canine species. Accordingly, an aspect of the invention is directed to a method of improving dental hygiene in a canine or to the use of a DNase, a mutanase, and a beta-glucanase for the preparation of composition for improving dental hygiene. Results showed that the enzyme composition containing mutanase, beta-glucanase and PDE had an 82.9% prevention effect on formation of canine multispecies biofilm. An aspect of the invention is directed to the prevention of canine oral diseases associated with a species of a bacteria selected from the group consisting of Neisseria, Pasteurella, Pseudomonas, Capnocytophaga and Enterococcus, particular selected from species selected from the group consisting of Neisseria zoodegmatis, Pasteurella canis, Pseudomonas fragi, Capnocytophaga canis and Enterococcus faecalis, more preferably selected from the group consisting of Neisseria zoodegmatis DSM21643, Pasteurella canis DSM22968, Pseudomonas fragi DSM3456, Capnocytophaga canis DSM101831 and Enterococcus faecalis DSM20478.

A further aspect of the invention is directed to the use of composition of the invention for the preparation of an oral care product for the prevention of plaque, gum disease and/or dental cavities in a canine and/or for the improvement of general oral hygiene in a canine.

### PREFERRED EMBODIMENTS I

1) An enzymatic oral care composition comprising a DNase, a mutanase, a beta-glucanase, and at least one oral care ingredient.
2) The oral care composition according to embodiment 1, wherein the DNase has at least 80% sequence identity to SEQ ID NO:1; preferably the DNase comprises, consists essentially of, or consists of SEQ ID NO:1.
3). The oral care composition according to any of embodiments 1-2, wherein the mutanase has at least 80% sequence identity to SEQ ID NO:2; preferably the mutanase comprises, consists essentially of, or consists of SEQ ID NO:2.
4) The oral care composition according to any of embodiments 1-3, wherein the beta-glucanase has at least 80% sequence identity to SEQ ID NO:3; preferably the DNase comprises, consists essentially of, or consists of SEQ ID NO:3.
5) The oral care composition according to any of embodiments 1-4, wherein at least one, e.g., at least two, or all, of the DNase, mutanase, and beta-glucanase has on par or improved chemical stability in the presence of at least one oral care ingredient selected from the group consisting of benzoate (preferably sodium benzoate), EDTA, ethanol, glycerol, phosphate (preferably sodium phosphate or potassium phosphate), fluoride (preferably sodium fluoride, sodium monofluorophosphate, calcium fluoride, or stannous fluoride), hydrogen peroxide, and mannitol.
6) The oral care composition according to any of embodiments 1-5 in the form of an internal oral care composition; preferably in the form of a toothpaste or toothpaste tablet, dental cream, mouthwash or mouthwash tablet, mouth rinse, lozenge, pastille, chewing gum, confectionary, or candy.
7) The oral care composition according to any of embodiments 1-5 in the form of an external oral care composition; preferably in the form of denture cleaning solution, denture cleaning tablet, or denture cleaning powder.
8) The oral care composition according to any of embodiments 1-7 for use as a medicament.
9) The oral care composition according to any of embodiments 1-7 for use in the treatment of oral disease; preferably for use in the treatment of periodontal disease and/or dental caries.
10) Use of an oral care composition according to any of embodiments 1-7 for treatment or prophylactic treatment of a human subject.
11) A method of treatment of a human subject, the method comprising administering an oral care composition according to any of embodiments 1-7; preferably the oral care composition is administered to the oral cavity of the human subject.
12) A method for removing oral biofilm, the method comprising contacting the oral biofilm with an oral care composition according to any of embodiments 1-7.
13) The method of embodiment 12, wherein the oral biofilm is located on an object, preferably a denture.
14) The method according to embodiment 13, wherein the denture is located inside or outside the oral cavity.
15) A kit of parts comprising:
   a) an oral care composition according to any of embodiments 1-7; and
   b) instructions for use.

### PREFERRED EMBODIMENTS II

1) An enzymatic oral care composition comprising a DNase, a mutanase, a beta-glucanase, and at least one oral care ingredient.
2) The oral care composition according to embodiment 1, wherein the DNase is a polypeptide having DNase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to SEQ ID NO:1.
3) The oral care composition according to embodiment 2, wherein the DNase has at least 80% sequence identity to SEQ ID NO:1; preferably the DNase comprises, consists essentially of, or consists of SEQ ID NO:1.
4) The oral care composition according to any of embodiments 1-3, wherein the mutanase is selected from the group consisting of:
   a) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:2;
   b) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:4;
   c) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:5;
   d) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:6;
   e) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:7;
   f) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:8;
   g) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:9;
   h) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:10;
   i) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:11;
   j) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:12;
   k) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:13; and
   l) a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:14.
5) The oral care composition according to any of embodiments 1-4, wherein the mutanase has at least 80% sequence identity to SEQ ID NO:2; preferably the mutanase comprises, consists essentially of, or consists of SEQ ID NO:2.
6) The oral care composition according to any of embodiments 1-5, wherein the beta-glucanase is selected from the group consisting of:
   a) a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:3;
   b) a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:15; and
   c) a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:16.
7) The oral care composition according to any of embodiments 1-6, wherein the beta-glucanase has at least 80% sequence identity to SEQ ID NO:3; preferably the beta-glucanase comprises, consists essentially of, or consists of SEQ ID NO:3.
8) The oral compositions according to any of embodiments 1-7,
   i) wherein the DNase is a polypeptide having DNase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to SEQ ID NO:1;
   ii) wherein the mutanase is a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:2; and
   iii) wherein the beta-glucanase is selected from the group consisting of:
   a) a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:3;
   b) a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:15; and
   c) a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:16.
9) The oral care composition according to any of embodiments 1-8,
   i) wherein the DNase is a polypeptide having DNase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to SEQ ID NO:1;
   ii) wherein the mutanase is a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:2; and
   iii) wherein the beta-glucanase a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:3.
10) The oral care composition according to any of embodiments 1-8,
   i) wherein the DNase is a polypeptide having DNase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to SEQ ID NO:1;
   ii) wherein the mutanase is a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:2; and
   iii) wherein the beta-glucanase is a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:15.
11) The oral care composition according to any of embodiments 1-8,
   i) wherein the DNase is a polypeptide having DNase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to SEQ ID NO:1;
   ii) wherein the mutanase is a polypeptide having endo-1,3-α-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:2; and
   iii) wherein the beta-glucanase is a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:16.
12) The oral care composition according to any of embodiments 1-8,
   i) wherein the DNase comprises, consists essentially of, or consists of SEQ ID NO:1;
   ii) wherein the mutanase comprises, consists essentially of, or consists of SEQ ID NO:2; and
   iii) wherein the beta-glucanase a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:3.
13) The oral care composition according to any of embodiments 1-8,
   i) wherein the DNase comprises, consists essentially of, or consists of SEQ ID NO:1;
   ii) wherein the mutanase comprises, consists essentially of, or consists of SEQ ID NO:2; and
   iii) wherein the beta-glucanase a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:15.
14) The oral care composition according to any of embodiments 1-8,
   i) wherein the DNase comprises, consists essentially of, or consists of SEQ ID NO:1;
   ii) wherein the mutanase comprises, consists essentially of, or consists of SEQ ID NO:2; and
   iii) wherein the beta-glucanase a polypeptide having endo-1,3(4)-β-glucanase activity and a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%, to the polypeptide of SEQ ID NO:16.
15) The oral care composition according to any of embodiments 1-8,
   i) wherein the DNase comprises, consists essentially of, or consists of SEQ ID NO:1;
   ii) wherein the mutanase comprises, consists essentially of, or consists of SEQ ID NO:2; and
   iii) wherein the beta-glucanase comprises, consists essentially of, or consists of SEQ ID NO:3.
16) The oral care composition according to any of embodiments 1-8,
   i) wherein the DNase comprises, consists essentially of, or consists of SEQ ID NO:1;
   ii) wherein the mutanase comprises, consists essentially of, or consists of SEQ ID NO:2; and
   iii) wherein the beta-glucanase comprises, consists essentially of, or consists of SEQ ID NO:15.
17) The oral care composition according to any of embodiments 1-8,
   i) wherein the DNase comprises, consists essentially of, or consists of SEQ ID NO:1;
   ii) wherein the mutanase comprises, consists essentially of, or consists of SEQ ID NO:2; and
   iii) wherein the beta-glucanase comprises, consists essentially of, or consists of SEQ ID NO:16.
18) The oral care composition according to any of embodiments 1-17, wherein at least one, e.g., at least two, or all, of the DNase, mutanase, and beta-glucanase has on par or improved chemical stability in the presence of at least one oral care ingredient selected from the group consisting of benzoate (preferably sodium benzoate), EDTA, ethanol, glycerol, phosphate (preferably sodium phosphate or potassium phosphate), fluoride (preferably sodium fluoride, sodium monofluorophosphate, calcium fluoride, or stannous fluoride), hydrogen peroxide, and mannitol.
19) The oral care composition according to embodiment 18, wherein chemical stability is determined according to Example 1 herein; preferably wherein chemical stability is determined as thermal stability defined by the thermal unfolding transition midpoint (Tm) in the presence of a particular oral care ingredient.
20) The oral care compositions according to any of embodiments 1-19, wherein the oral care composition provides improved biofilm prevention compared to Composition B as determined according to Example 2 herein.
21) The oral care composition according to any of embodiments 1-20 in the form of an internal oral care composition; preferably in the form of a toothpaste or toothpaste tablet, dental cream, mouthwash or mouthwash tablet, mouth rinse, lozenge, pastille, chewing gum, confectionary, or candy; more preferably in the form of a toothpaste tablet, mouthwash tablet, lozenge, pastille, chewing gum, confectionary or candy; most preferably in the form of a toothpaste tablet, lozenge, or chewing gum.
22) The oral care composition according to any of embodiments 1-20 in the form of an external oral care composition; preferably in the form of denture cleaning solution, denture cleaning tablet, or denture cleaning powder.
23) The oral care composition according to any of embodiments 1-22 for use as a medicament.
24) The oral care composition according to any of embodiments 1-22 for use in the treatment of oral disease; preferably for use in the treatment of periodontal disease and/or dental caries.
25) The oral care composition according to any of embodiments 1-22 for use in prevention and/or removal of oral biofilm.
26) Use of an oral care composition according to any of embodiments 1-22 for treatment or prophylactic treatment of a human subject.
27) Use of an oral care composition according to any of embodiments 1-22 for altering (e.g., improving) dental plaque morphology.
28) Use of an oral care composition according to any of embodiments 1-22 for increasing microbial richness of the oral microbiome.
29) Use of an oral care composition according to any of embodiments 1-22 for preventing and/or removing oral biofilm.
30) A method of treatment of a human subject, the method comprising administering an oral care composition according to any of embodiments 1-22; preferably the oral care composition is administered to the oral cavity of the human subject.
31) A method for removing oral biofilm, the method comprising contacting the oral biofilm with an oral care composition according to any of embodiments 1-22.
32) The method of embodiment 31, wherein the oral biofilm is located on an object, preferably a denture.
33) The method according to embodiment 32, wherein the denture is located inside or outside the oral cavity.
34) A kit of parts comprising:
   a) an oral care composition according to any of embodiments 1-22; and
   b) instructions for use.

### EXAMPLES

### Activity Assays

### DNase Activity Assay 1

DNase activity may be determined on DNase Test Agar with Methyl Green (BD, Franklin Lakes, NJ, USA), which is prepared according to the supplier's instructions. Briefly, 21 g of agar is dissolved in 500 ml water and then autoclaved for 15 min at 121° C. Autoclaved agar is then tempered to 48° C. in water bath, and 20 ml of agar is poured into petri dishes with and allowed to solidify by incubation overnight at room temperature. On solidified agar plates, 5 μl of enzyme solutions is added and DNase activity is observed as colorless zones around the spotted enzyme solutions.

### DNase Activity Assay 2

DNase activity may be determined using the DNaseAlert Kit (11-02-01-04, IDT Integrated DNA Technologies) according to the supplier's instructions. Briefly, 95 μl DNase sample is mixed with 5 μl substrate in a microtiter plate, and fluorescence is immediately measured, e.g., using a Clariostar microtiter reader from BMG Labtech (536 nm excitation, 556 nm emission).

Mutanase Activity Assay Mutanases produce reducing sugars upon hydrolysis of a mutan substrate, and the release of reducing sugars may be detected and used to determine endo-1,3-α-glucanase activity. Mutan substrate may be prepared according to the procedure described in Fuglsang et al., J. Biol. Chem., 2000, vol. 375, no. 3, pp. 2009-2018.

Mutan substrate is dissolved in 0.5% of DMSO to a final concentration of 60 ppm (0.006%), and mutanases are diluted to a stock concentration of 200 ppm (0.2 mg/mL). The reaction is initiated by incubating 60 ppm mutanase (0.06 mg/mL) together with 100 μL pre-heated mutan substate in 0.1 M sodium acetate buffer (pH 5.6) in a clear bottom 96-well microtiter plate (ThermoFisher Scientific) for 10 minutes at 50° C. and 300 rpm. Samples without enzyme addition are used as substrate controls.

The mutan hydrolysis reaction is quenced by adding 80 μL alkaline reagent containing potassium sodium tartrate (50 g/L), PAHBAH (20 g/L, CAS No.: 5351-23-5), bismuth(II) acetate (5.52 g/L) and sodium hydroxide (0.5 M), and the quenched samples are incubated for an additional 20 minutes at 50° C. and 300 rpm.

Mutanase activity may be determined by measuring absorbance at 405 nm.

### Beta-Glucanase Activity Assay

Endo-1,3(4)-β-glucanase activity may be determined by a reducing end assay in the following way:

20 μL of a beta-glucanase stock solution (obtained, e.g., by adding 100 uL Viscozyme® L to 10 ml MilliQ water containing 0.01% Triton-X) and 180 μL beta-glucan substrate solution (obtained, e.g., by dissolving 12.5 mg beta-glucan, e.g., beta-glucan from barley, medium viscosity, catalog no. P-BGBM, Megazyme, in 5 ml B&R buffer containing 50 mM boric acid, 50 mM acetic acid, 50 mM KCl, 1 mM CaCl₂, and 0.01% Triton-X in MilliQ water) are mixed, and the enzyme-substrate mixture is incubated for 30 min at 30° C.

Following incubation, 20 μL of the enzyme-substrate mixture is mixed with 130 μL MilliQ water and 70 μL PAHBAH reagent (obtained by dissolving 0.375 g 4-hydroxy-benzhydrazide in 25 ml K-Na-tartrate solution, which is obtained by dissolving 50 g K-Na-tartrate and 20 g NaOH in 1 L MilliQ water). This solution is then incubated at 95° C. for 10 min, followed by cooling on ice for 1 min.

Endo-1,3(4)-β-glucanase activity may then be determined by measuring the absorbance at 410 nm.

### Enzyme Compositions

In the present examples, the following enzyme compositions are evaluated:

| Name | Enzyme content |
|---|---|
| Composition A | DNase (SEQ ID NO: 1), 60 ppm |
| | Mutanase (SEQ ID NO: 2), 60 ppm |
| | Viscozyme ® L(*), 60 ppm |
| Composition B | DNase (SEQ ID NO: 1), 60 ppm |
| | Mutanase (SEQ ID NO: 2), 60 ppm |
| Composition C | DNase (SEQ ID NO: 1), 60 ppm |
| | Mutanase (SEQ ID NO: 2), 60 ppm |
| | Beta-glucanase (SEQ ID NO: 3), 60 ppm |
| Composition D | DNase (SEQ ID NO: 1), 60 ppm |
| | Mutanase (SEQ ID NO: 2), 60 ppm |
| | Beta-glucanase (SEQ ID NO: 15), 60 ppm |
| Composition E | DNase (SEQ ID NO: 1), 60 ppm |
| | Mutanase (SEQ ID NO: 2), 60 ppm |
| | Beta-glucanase (SEQ ID NO: 16), 60 ppm |

(*)Viscozyme ® L (Novozymes A/S) is a commercially available multi-component enzyme composition derived from Aspergillus aculeatus. The main enzymatic activity of Viscozyme ® L is beta-glucanase activity provided by its main component (SEQ ID NO: 3), but Viscozyme ® L also contains other beta-glucanases such as SEQ ID NO: 15 and SEQ ID NO: 16. Viscozyme ® L is standardized to 100 FBG/g and has a density of approximately 1.2 g/mL.

### Example 1: Thermal Stability of SEQ ID NOs:1-4, 6, and 11 in Presence of Oral Care Ingredients

### Preparation of Oral Care Formulations for Thermal Stability Measurements

The thermal stability or thermal unfolding transition midpoint (Tm) of DNase (SEQ ID NO: 1), mutanase (SEQ ID NOs:2, 4, 6, and 11), and beta-glucanase (SEQ ID NO:3) was measured in the presence of widely used oral care ingredients within concentration ranges commonly used in oral care product formulations and selected oral care commercial products. The Tm parameter is used to evaluate the thermal stabilities as this is the temperature at which there are equal populations of folded and unfolded protein molecules. Tm is a widely accepted parameter to use when evaluating thermal stability.

Highly pure and biotechnology grade reagents were obtained from various suppliers and stock solutions were freshly prepared using MilliQ water. These formulation chemicals and their stock as well as the final concentrations used in the Tm measurement are listed in the Table 1.

Purified preparations of the enzyme samples were diluted to a stock concentration of 2 mg/ml prior to a further 10 times dilution in the oral care formulations consisting of individual oral care ingredients (concentrations specified in Tables 2-5), citrate phosphate buffer (McIlvaine buffer, see below) and MilliQ water corresponding to a final protein concentration of 0.2 mg/ml. Using robotics arm, all dilutions were made in a 384-well small volume deep well plate (Greiner Bio-One International, item number 784201) with a final volume of 70 μl and used for thermal stability measurements. Tm measurements for each enzyme were performed close to the physiological pH range of the oral cavity using McIlvaine buffer at pH 5.0 and pH 6.0. 100 ml of McIlvaine buffer pH 5.0 was prepared by mixing 51.50 ml 0.2 M Na₂HPOₐ+48.50 ml 0.1 M citric acid whereas pH 6.0 McIlvaine buffer prepared by mixing 63.15 ml 0.2 M Na₂HPOₐ+36.85 ml 0.1 M citric acid.

TABLE 1
Reagents

| | | Product | Stock |
|---|---|---|---|
| Chemicals | Source | number | conc. (mM) |
| Sodium benzoate | Sigma | 18106 | 100 |
| EDTA | Sigma | E5134 | 10 |
| Ethanol | VWR Chemicals | 20824.296 | 10000 |
| Glycerol | Sigma | G5516 | 7600 |
| Sodium phosphate | Sigma | 71643 | 50 |
| Sorbitol | Sigma | S6021 | 3000 |
| Potassium sorbate | Sigma | 85520 | 100 |
| Sodium fluoride | Sigma | 919 | 100 |
| Hydrogen peroxide | Sigma | H1009 | 1000 |
| Mannitol | Sigma | M9546 | 1000 |

### Thermal Stability in Oral Care Formulations

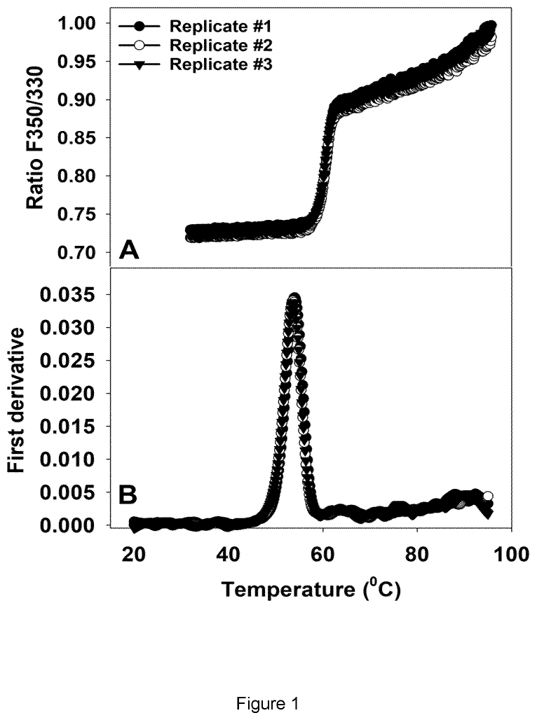

Thermal stability measurements were performed using a capillary based nano differential scanning fluorescence instrument (nanoDSF); Prometheus NT.Plex (NanoTemper Technologies GmbH, Munhchen, Germany). Standard nanoDSF grade capillary chips were used (Cat #: PR-AC002) from NanoTemper Technologies. The protein samples were loaded into the capillaries (each sample in triplicate) by capillary action. The emission intensities at 330 and 350 nm were optimized by altering the LED power on the instrument to ensure sufficient signal. The fluorescence signals at 330 and 350 nm were monitored continuously as a function of temperature (heating rate used for thermal unfolding was 3.3° C. per minute from 20° C. to 95° C.). The data was analyzed using the PR.StabilityAnalysis_1.1.0.11077 software provided by the manufacturer. The analysis is model independent and simply takes the peak maximum of the first derivative which corresponds to the approximate thermal unfolding transition midpoint, defined as Tm (see FIG. 1).

### Reproducibility of Thermal Stability Data

FIG. 1 shows an example of the thermal stability data generated using the nanoDSF instrument. Panel A is an example of the data obtained (the ratio of the fluorescence emission at 350 nm to 330 nm) in triplicate for SEQ ID NO:2 as a function of temperature. Panel B shows the first derivative of the raw data in Panel A. The peak maximum in the first derivative plot corresponds to the mid-point of the thermal unfolding transition, referred to as Tm. In this example, Tm corresponds to 53.6° C. at pH 6.0 and is highly reproducible within the three replicates.

The data shown in FIG. 1 is an example of the type of data that was generated for all enzymes in the different formulations using nanoDSF. In all cases, the data showed a clear unfolding transition, and a clearly defined peak in the first derivative, and were highly reproducible.

### Results

Table 2 and Table 3 shows the average Tm values of SEQ ID NO:1 (DNase), SEQ ID NO:2 (mutanase), and SEQ ID NO:3 (beta-glucanase) originating from triplicate measurements at pH 5 and pH 6, respectively. Table 4 and Table 5 shows the average Tm values of SEQ ID NO:4 (mutanase), SEQ ID NO:6 (mutanase), and SEQ ID NO:11 (mutanase) originating from triplicate measurements at pH 5 and pH 6, respectively. Table 6 and Table 7 shows the average Tm values of SEQ ID NO:15 (beta-glucanase) and SEQ ID NO:16 (beta-glucanase) originating from triplicate measurements at pH 5 and pH 6, respectively.

From the data shown in the Tables 2-7, it is clear that the tested enzymes have on par or improved thermal stability in the presence of a number of formulation ingredients used in oral care. In this context, the term “on par thermal stability” means that the Tm value of an enzyme co-formulated with an oral care component is within +/−5% of the Tm value of the same enzyme alone (i.e., the control), and the term “improved thermal stability” means that the Tm value of an enzyme co-formulated with an oral care component is increased more than 5%, e.g., 10%, 15%, 20%, 25%, 30%, 40%, 50%, 60%, 70%, 80%, 90%, 100%, or even more, compared to the Tm value of the same enzyme alone (i.e., the control).

Taken together, the data presented in this example clearly shows that the evaluated enzymes have high thermal stabilities in the presence of widely used oral care components, making them compatible with common oral care formulations.

TABLE 2
Tm values of SEQ ID NOs: 1-3 at pH 5

| | | Tm (° C.) at pH 5.0 | | |
|---|---|---|---|---|
| | | | | SEQ ID NO: 3 |
| | Conc. | SEQ ID NO: 1 | SEQ ID NO: 2 | (Beta- |
| Ingredient | (mM) | (DNase) | (Mutanase) | glucanase) |
| Control | 0 | 60.1 | 58.7 | 56.2 |
| EDTA | 1 | 61.9 | 54.3 | 56.2 |
| Ethanol | 1000 | 58.5 | 58.1 | 63.4 |
| Glycerol | 1000 | 63.8 | 55.3 | 65.5 |
| | 3250 | 68.6 | 63.6 | 68.7 |
| Hydrogen peroxide | 100 | 58.8 | 52.3 | 64.1 |
| | 500 | 58.9 | 44.7 | 63.3 |
| Potassium sorbate | 10 | 60.7 | 52.7 | 62.0 |
| | 35 | 57.9 | 54.7 | 57.6 |
| Mannitol | 250 | 60.4 | 59.9 | 56.7 |
| | 550 | 64.2 | 56.1 | 57.6 |
| Sodium benzoate | 10 | 61.4 | 53.3 | 56.2 |
| | 35 | 60.6 | 52.5 | 63.2 |
| Sodium fluoride | 20 | 61.8 | 53.6 | 56.4 |
| | 40 | 60.1 | 58.5 | 56.5 |
| Sodium phosphate | 5 | 61.4 | 50.4 | 56.2 |
| | 10 | 61.0 | 48.3 | 54.1 |
| Sorbitol | 550 | 64.0 | 56.0 | 57.8 |
| | 2200 | 71.9 | 64.5 | 65.5 |

TABLE 3
Tm values of SEQ ID NOs: 1-3 at pH 6

| | | Tm (° C.) at pH 6.0 | | |
|---|---|---|---|---|
| | | | | Beta- |
| | Conc. | DNase | Mutanase | glucanase |
| Ingredient | (mM) | (SEQ ID NO: 1) | (SEQ ID NO: 2) | (SEQ ID NO: 3) |
| Control | 0 | 61.9 | 53.6 | 55.5 |
| EDTA | 1 | 59.9 | 59.0 | 56.1 |
| Ethanol | 1000 | 60.6 | 52.6 | 54.1 |
| Glycerol | 1000 | 62.5 | 60.3 | 56.3 |
| | 3250 | 68.6 | 58.7 | 59.2 |
| Hydrogen peroxide | 100 | 60.5 | 47.6 | 54.8 |
| | 500 | 56.2 | 49.1 | 54.8 |
| Potassium sorbate | 10 | 59.9 | 57.4 | 54.9 |
| | 35 | 58.7 | 50.5 | 53.7 |
| Mannitol | 250 | 62.9 | 54.8 | 56.4 |
| | 550 | 62.7 | 60.8 | 58.4 |
| Sodium benzoate | 10 | 59.6 | 58.2 | 55.3 |
| | 35 | 58.4 | 56.4 | 55.2 |
| Sodium fluoride | 20 | 60.2 | 59.0 | 55.6 |
| | 40 | 61.9 | 53.7 | 55.3 |
| Sodium phosphate | 5 | 61.9 | 54.6 | 53.8 |
| | 10 | 61.2 | 51.2 | 53.7 |
| Sorbitol | 550 | 62.5 | 60.9 | 58.0 |
| | 2200 | 71.1 | 68.2 | 68.0 |

TABLE 4
Tm values of SEQ ID NOs: 4, 6, and 11 at pH 5

| | | Tm (° C.) at pH 5 | | |
|---|---|---|---|---|
| | Conc. | SEQ ID NO: 4 | SEQ ID NO: 6 | SEQ ID NO: 11 |
| Ingredient | (mM) | (Mutanase | (Mutanase) | (Mutanase) |
| Control | 0 | 66.5 | 62.9 | 61.8 |
| EDTA | 1 | 66.4 | 63.0 | — |
| Ethanol | 1000 | 65.8 | 61.6 | 61.7 |
| Glycerol | 1000 | 67.8 | 63.8 | 62.9 |
| | 3250 | 71.1 | 66.1 | 64.3 |
| Hydrogen peroxide | 100 | 64.4 | 57.2 | 61.1 |
| | 500 | 58.7 | 54.3 | 59.2 |
| Potassium sorbate | 10 | 67.1 | 61.5 | 61.8 |
| | 35 | 65.6 | 59.6 | 61.6 |
| Mannitol | 250 | 67.2 | 63.6 | 62.6 |
| | 550 | 68.5 | 64.4 | 63.4 |
| Sodium benzoate | 10 | 66.0 | 62.3 | 61.6 |
| | 35 | 65.5 | 61.0 | 61.2 |
| Sodium fluoride | 20 | 66.4 | 62.9 | 61.9 |
| | 40 | 66.6 | 62.8 | 62.1 |
| Sodium phosphate | 5 | 69.0 | 58.3 | 61.6 |
| | 10 | 69.6 | 55.6 | 60.8 |
| Sorbitol | 550 | 67.8 | 64.1 | 63.3 |
| | 2200 | 74.6 | 70.5 | 68.7 |

TABLE 5
Tm values of SEQ ID NOs: 4, 6, and 11 at pH 6

| | | Tm (° C.) at pH 6 | | |
|---|---|---|---|---|
| | Conc. | SEQ ID NO: 4 | SEQ ID NO: 6 | SEQ ID NO: 11 |
| Ingredient | (mM) | (Mutanase) | (Mutanase) | (Mutanase) |
| Control | 0 | 69.6 | 57.9 | 61.4 |
| EDTA | 1 | 69.5 | 57.9 | 59.9 |
| Ethanol | 1000 | 68.9 | 56.2 | 61.1 |
| Glycerol | 1000 | 71.1 | 58.6 | 62.1 |
| | 3250 | 75.1 | 61.2 | 63.2 |
| Hydrogen peroxide | 100 | 67.3 | 51.4 | 60.9 |
| | 500 | 59.9 | 49.6 | 59.3 |
| Potassium sorbate | 10 | 69.0 | 57.1 | 61.1 |
| | 35 | 66.8 | 55.9 | 60.6 |
| Mannitol | 250 | 70.2 | 58.5 | 62.0 |
| | 550 | 71.2 | 60.1 | 62.9 |
| Sodium benzoate | 10 | 69.2 | 57.2 | 61.1 |
| | 35 | 68.2 | 56.6 | 60.4 |
| Sodium fluoride | 20 | 69.5 | 57.4 | 61.4 |
| | 40 | 69.5 | 57.4 | 61.4 |
| Sodium phosphate | 5 | 69.7 | 54.8 | 60.4 |
| | 10 | 69.7 | 53.0 | 59.6 |
| Sorbitol | 550 | 71.2 | 59.2 | 62.9 |
| | 2200 | 77.7 | 65.9 | 68.0 |

TABLE 6
Tm values of SEQ ID NO: 15 and SEQ ID NO: 16 at pH 5

| | | Tm (° C.) at pH 5 | |
|---|---|---|---|
| | Conc. | SEQ ID NO: 15 | SEQ ID NO: 16 |
| Ingredient | (mM) | (Beta-glucanase) | (Beta-glucanase) |
| Control | 0 | 71.0 | 82.7 |
| EDTA | 1 | 71.2 | 82.7 |
| Ethanol | 1000 | 70.3 | 81.4 |
| Glycerol | 1000 | 71.9 | 83.1 |
| | 3250 | 73.0 | 84.6 |
| Potassium sorbate | 10 | 70.9 | 82.0 |
| | 35 | 69.8 | 80.7 |
| Mannitol | 250 | 71.9 | 83.2 |
| | 550 | 72.5 | 83.8 |
| Sodium benzoate | 10 | 71.2 | 82.3 |
| | 35 | 70.8 | 81.5 |
| Sodium fluoride | 20 | 71.3 | 82.8 |
| | 40 | 71.2 | 82.7 |
| Sorbitol | 550 | 72.6 | 83.7 |
| | 2200 | 76.9 | 87.6 |

TABLE 7
Tm values of SEQ ID NO: 15 and SEQ ID NO: 16 at pH 6

| | | Tm (° C.) at pH 6 | |
|---|---|---|---|
| | Conc. | SEQ ID NO: 15 | SEQ ID NO: 16 |
| Ingredient | (mM) | (Beta-glucanase) | (Beta-glucanase) |
| Control | 0 | 67.7 | 78.4 |
| EDTA | 1 | 68.6 | 79.2 |
| Ethanol | 1000 | 67.0 | 76.8 |
| Glycerol | 1000 | 68.8 | 79.7 |
| | 3250 | 70.9 | 82.3 |
| Potassium sorbate | 10 | 67.5 | 78.3 |
| | 35 | 66.6 | 77.7 |
| Mannitol | 250 | 68.5 | 79.2 |
| | 550 | 69.3 | 80.0 |
| Sodium benzoate | 10 | 67.6 | 78.5 |
| | 35 | 67.4 | 78.3 |
| Sodium fluoride | 20 | 67.8 | 78.5 |
| | 40 | 67.7 | 78.6 |
| Sorbitol | 550 | 69.3 | 79.9 |
| | 2200 | 74.6 | 84.6 |

### Example 2: Human Saliva Biofilm Prevention Assay

A human saliva biofilm prevention assay was carried out using the method described in WO 2020/099490 with a few modifications. Briefly, biofilm was grown in 96-well plates, and enzyme-treated samples were made by incubation with Composition A in citrate-phosphate buffer (McIlvaine buffer, pH 6, prepared by mixing 12.63 ml 0.2 M Na₂HPO₄+7.37 ml 0.1 M citric acid). As comparator, Composition B in the same citrate-phosphate buffer was also evaluated. Incubation was conducted at 37° C. for 24 hours without shaking in a ThermoFisher Scientific™ Rectangular AnaeroBox™ container under microaerophilic conditions (ThermoScientific AnaeroGen 2,5L #AN0025A). Buffer-treated samples made by incubation with McIlvaine buffer, pH 6, were included as controls. All samples were evaluated in eight replicates.

After incubation, planktonic bacteria were removed by two gentle washes with 100 μl 0.9% sodium chloride and biofilms were stained with 0.95% crystal violet solution for 15 min at room temperature. Plates were rinsed twice with 100 μL 0.9% sodium chloride and adhered dye was dissolved with a solution of 96% ethanol and 0.1% acetic acid in water. Absorbance was measured at 600 nm with a SpectraMax M3 microplate reader (Molecular Devices).

For the data processing, the absorbance was taken to be proportional to the extent of remaining biofilm after enzyme or buffer treatment. The results were expressed as percentage of biofilm prevention and was calculated as follows: 100−((A600 nm enzyme-treated sample)/(A600 nm buffer-treated sample)×100), where A600 nm refers to the average of eight absorbance measurements conducted at 600 nm of either enzyme-treated or buffer-treated samples.

As can be seen from Table 8, Composition A comprising DNase, mutanase, and beta-glucanase exhibits an enhanced biofilm prevention effect compared to Composition B comprising only DNase and mutanase (87.3% vs. 79.0%).

TABLE 8
Oral biofilm prevention.

| | Biofilm | Standard |
|---|---|---|
| Enzyme composition | prevention | error |
| Composition A (DNase + mutanase + Viscozyme ® L) | 87.3% | 0.5 |
| Composition B (DNase + mutanase) | 79.0% | 0.7 |
| Composition C (DNase + mutanase + beta-glucanase) | 84.9% | 0.4 |
| Composition D (DNase + mutanase + beta-glucanase) | 85.8% | 0.2 |
| Composition E (DNase + mutanase + beta-glucanase) | 85.7% | 0.3 |

### Example 3: Biofilm Removal Assays

### Composition A

Biofilm removal with Compositions A was assessed using two types of biofilms: a multi-species biofilm grown from human saliva and a single-species biofilm grown from Streptococcus mutans. The two types of biofilms were grown in 384/1536-well microplates with black well walls and an optically cyclic olefin bottom in the following way:

Multi-species saliva biofilm: human saliva was collected from 12 different volunteers. The collected saliva was diluted to 50% in equal volumes of PBS and glycerol, then frozen and stored at −20° C. until use. To form the biofilm, 8 μl non-sterilized saliva was added to each well, along with 8 μl sterilized saliva, 6.4 μl sucrose and 57.6 μl Brain Heart Infusion (BHI) broth (Sigma, product no. 53286). The plates were inoculated for 16 h at 37° C. and 50 rpm.

Single-species S. mutans biofilm: 80 μl of Tripticase Soy Broth (TSB)+1% sucrose containing 1×10⁷ CFU/ml bacterial inoculum (based on a McFarland solution of S. mutans UA159) was added to each well. The plates were inoculated for 16 h at 37° C. under anaerobic conditions.

Following incubation, the liquid was then removed via pipetting from the grown biofilms and replaced by 80 μl of Composition A in citrate-phosphate buffer (McIlvaine buffer, pH 6). The plates were incubated for 0.5 h (saliva biofilm) or 2 h (S. mutans biofilm) at 37° C. and 50 rpm.

Composition A in citrate phosphate buffer (McIlvaine buffer, pH 6) was then removed by turning the plates upside down. The biofilm was washed once with 80 μl of 0.9% aqueous NaCl (added and aspirated) and partially dried by turning the plate upside down and tapping it gently face down to remove any droplets left in the wells.

Biofilm removal was assessed using an Opera Phenix™ high content screening system (PerkinElmer) equipped with a 63× water immersion objective after staining with wheat germ agglutinin-Alexa Fluor™ 633 conjugate (WGA) (excitation length: 640 nm; emission length: 650-760 nm), FITC (excitation length: 488 nm; emission length: 500-550 nm) and Syto™ 85 (excitation length: 561 nm; emission length: 570-630 nm). All fields of each well were imaged to evaluate biofilm removal performance. Harmony software was used for data processing.

As can be seen from Table 9a, Composition A (comprising DNase, mutanase, and Viscozyme® L) exhibits a potent biofilm removal effect, in particular with respect to biofilm grown from S. mutans.

TABLE 9a
Oral biofilm removal.

| | | % Removal | % Removal | % Removal |
|---|---|---|---|---|
| Enzyme | | (EPS labelled | (EPS labelled | (eDNA labelled |
| composition | Biofilm model | with FITC) | with WGA) | with Syto ™ 85) |
| Composition A | Multi-species | 41.0 | 40.8 | 39.6 |
| Composition A | Single-species | 99.1 | 99.6 | 96.6 |

EPS = exopolysaccharide; eDNA = extracellular DNA.

### Compositions B, D, and E

Biofilm removal with Compositions B, D, and E was assessed using a multi-species biofilm grown from human saliva in a 96-well microtiter plate formate according to the method described in WO 2020/099490 with some modifications. Briefly, biofilm was grown for 24 hours and treated with either 200 μL McIlvaine buffer pH 6 (control; prepared by mixing 12.63 ml 0.2 M Na₂HPO₄+7.37 ml 0.1 M citric acid) or Composition C, D, or E in McIlvaine buffer, pH 6, for 30 minutes at 37° C. with agitation (50 rpm). All samples were evaluated in eight replicates.

After enzyme treatment, samples were rinsed twice with 200 μL 0.9% sodium chloride and stained with 0.095% crystal violet solution for 15 minutes at room temperature. Samples were rinsed three times with 200 μL 0.9% sodium chloride and the dye was extracted with 33% acetic acid solution. Absorbance was measured at 600 nm with a SpectraMax M3 microplate reader (Molecular Devices).

For the data processing, the absorbance was taken to be proportional to the extent of remaining biofilm after enzyme or buffer treatment. The percentage of biofilm removal was calculated as follows: 100−((A600 nm enzyme-treated sample)/(A600 nm buffer-treated sample)×100), where A600 nm refers to the average of eight absorbance measurements conducted at 600 nm of either enzyme-treated or buffer-treated samples.

As can be seen from Table 9b, Compositions D and E (comprising DNase, mutanase, and beta-glucanase) exhibits a potent biofilm removal effect that is improved compared to Composition B (comprising DNase and mutanase).

TABLE 9b
Oral biofilm removal.

| | Biofilm | Standard |
|---|---|---|
| Enzyme composition | removal | error |
| Composition B (DNase + mutanase) | 29.7% | 8.3 |
| Composition D (DNase + mutanase + beta-glucanase) | 44.0% | 6.0 |
| Composition E (DNase + mutanase + beta-glucanase) | 41.5% | 6.1 |

### Example 4: Clinical Evaluation of the Effect of Composition a on Dental Biofilm and Salivary Microbiome in Patients with Fixed Orthodontic Appliances

The aim of the clinical study was to evaluate the effect of Composition A in a mouthwash format on the amount of dental biofilm and composition of the salivary microbiome in patients undergoing treatment with fixed orthodontic appliances.

### Materials and Methods

### Study Design, Ethical Approval and Registration:

The study employed a randomized, double-blind placebo-controlled design with two parallel arms. The project was approved by the Swedish ethical review authority, Sweden (Dnr 2020-05221) and registered in Clinical Trials.gov Identifier (NCT05033015).

### Study Population:

We invited 60 eligible patients (14-18 years) at the Orthodontic Specialist Clinic in Karlshamn, Sweden to enter the study. The subjects were consecutively enrolled, and the inclusion criteria were i) being under treatment with fixed orthodontic appliances (uni- or bi-maxillary) for at least 3-6 months and ii) visible biofilm accumulation around the bracket bases of at least 6 teeth. Patients with caries lesions, periodontal disease and/or soft tissue pathology were not invited. Likewise, we excluded patients with known food allergies or a history of allergies to ingredients in the test product, including allergy against enzymes. Thirty-five patients accepted the invitation and obtained verbal and written information about the project. We collected a signed consent from each patient and the custodians. The subjects were randomly allocated to the test- or placebo group after the baseline registrations by opening a computer-generated numbered envelope. For various Covid-19 related reasons, only 29 subjects completed the study protocol, giving a dropout rate of 18%.

### Intervention and Experimental Products:

We provided all subjects with a standardized enzyme-free toothpaste with 1450 ppm sodium fluoride to be used twice daily during the course of the study. After the baseline clinical registration, we asked the participants to rinse their mouth thoroughly by swishing 10 mL of the assigned liquid around the teeth morning and evening for 30 seconds. After rinsing, the subjects should spit out, avoid water rinsing and most importantly, we requested them to separate tooth brushing from mouth washing by at least 30 minutes. The duration of the intervention was 8 days (16 rinses). The experimental mouthwash contained Composition A in an aqueous solution as shown in Table 10. The placebo mouthwash had an identical taste and color but contained no enzymes. To secure blinding, the products came in color-coded tubes, each containing exactly 10 mL of the product. The clinical investigator sent a twice-daily SMS (morning and evening) reminder (Servicewell, Lund, Sweden) to the patients in order to enhance compliance. An independent university-based monitor guaranteed the allocation concealment.

TABLE 10
Composition of experimental mouthwash

| | Concentration in the |
|---|---|
| Mouthwash Ingredient | sample % (w/w) |
| DNase (SEQ ID NO: 1) | 60 ppm (0.006%) |
| Mutanase (SEQ ID NO: 2) | 60 ppm (0.006%) |
| Viscozyme ® L | 60 ppm (0.006%) |
| Sorbitol | 40.0% |
| Sodium citrate | 0.45% |
| Monobasic - Sodium phosphate | 1.04% |
| Potassium sorbate | 0.20% |
| Mint flavor | 0.10% |
| Water | 58.21% |

### Clinical Registrations and Endpoints:

The primary outcome was the amount of dental biofilm around the orthodontic brackets and a single calibrated examiner performed the registrations at baseline and after 8 days. To visualize the biofilm, teeth were illuminated with a violet UV-light in detection mode (D-Light Pro, GC Europe). The biofilm was then scored according to Beberhold and co-workers (Beberhold et al., Orthodontics (Chic.), 2012; 13:94-99): score 0=brackets are plaque free; score 1=isolated plaque islands on one tooth surface at the bracket base; score 2=plaque on two tooth surfaces at the bracket base; score 3=plaque on three tooth surfaces at the bracket base; and score 4=plaque on all tooth surfaces at the bracket base and/or gingival inflammation. The buccal surfaces of the upper and lower anterior teeth (incisors and cuspids) and premolars were registered. We divided the total sum of the scores with the number of teeth to express the Orthodontic Plaque Index (OPI) on patient level. The principal examiner scored the buccal mucosa along the gingival margin as 0=normal gingiva (bleak and firm), or 1=presence of gingivitis (red, swollen mucosa, bleeding on probing). We evaluated the inter-examiner reliability of the OPI on 10 teenagers under treatment with fixed orthodontic appliances, not taking part in the pilot study. Two trained clinicians assessed the biofilm scores independently at the bracket base of totally 178 teeth.

### Power Calculation and Statistical Methods for Clinical Data:

Due to the lack of previous clinical data with respect to the experimental mouthwash, we anticipated that a mean difference of 0.5 OPI units (SD 0.4) would be clinically meaningful. A calculation with α=0.05 and β=0.8 indicated that 18 patients in each group should be enrolled in the study. We processed the clinical data with the IBM-SPSS software (version 27.0, Chicago, USA). For continues data, differences between groups on subject level were compared with unpaired t-tests. Inter-examiner reliability was calculated and expressed with Cohen's kappa value. A p-value less than 0.05 was regarded as statistically significant. We performed the statistical calculations before the group allocation was unveiled.

### Saliva Sampling and Analyses:

The examiner collected unstimulated whole saliva samples at baseline and after eight days before the biofilm scoring. The subjects rinsed with tap water, accumulated saliva in their mouth and expectorated in a capped plastic tube (approximately 1.0 mL). The tube was immediately frozen and stored at −18° C. until transportation and further processing at Novozymes A/S in Denmark.

All samples were heat inactivated for 15 min at 95° C. followed by DNA extraction using the Macherey-Nagel™ NucleoSpin™ Soil kit according to the manufacturer's protocol. 600-800 μl of saliva sample was mixed with 850 μl PBS buffer and centrifuged at 10,000 rpm for 2 min. The lysis buffer was added directly to the pellet and transferred to the beads tube. The V3-V4 region of 16S was PCR amplified using universal primers and sequenced using Illumina MiSeq 300 bp paired end. We included negative control samples (no material/DNA) as well as positive controls consisting of a known mock community to validate the analyses.

### Bioinformatics and Statistical Analysis of the Saliva Microbiome Samples:

We used the USEARCH pipeline to generate zero radius operational taxonomic units (OTUs; Edgar, Nat. Methods., 2013; 10:996-998) using the same setting as described by Larsen et al. (Gut Microbes, 2021, 13(1), 1988836). The taxonomic classification of the unique filtered amplicons was conducted with the QIIME feature-classifier classify-sklearn (Bolyen et al., Nat. Biotechnol. 2019, vol. 37, pp. 852-857) and the Human Oral Microbiome Database v15.22 (Chen et al., 2010). All samples were rarefied to a read count of 10000. For the global composition of the salivary microbiome at baseline, we applied the Adonis Permanova test on the dissimilarity matrix using the Bray-Curtis metric (Bray and Curtis, Monographs, 1957; 27:326-349). To investigate if the mouthwash introduced a change in the global microbiome composition over time, the Bray-Curtis dissimilarity between baseline and follow-up samples for each individual was tested using a paired t-test. We established a linear mixed model to test effects of the mouthwash compared to the placebo group on microbial richness from baseline to follow up. The null hypothesis was that the change in richness between baseline and follow up for the test group was equal to the change in the placebo group. We used a similar model to test for significant changes for all OTUs present in more than 32 samples, where each OTU count was log transformed. We corrected all p-values for multiple testing according to Benjamini and Hochberg (J R Stat Soc Series, B. 1995; 57:289-300).

### Results

### Baseline Data:

The mean age of the patients was 15.4 years in the test group and 16.8 years in the placebo group. The most common orthodontic treatment indication was moderate to severe crowded dental arches and 18 patients had 2-4 premolars extracted. All patients had pre-adjusted fixed orthodontic appliances (0.022 slot size, MBT prescription, 3M™ Victory Series™ Low Profile Brackets, Unitek, CA, USA) in the maxilla and 24 were under treatment with bi-maxillary braces.

### Clinical Findings:

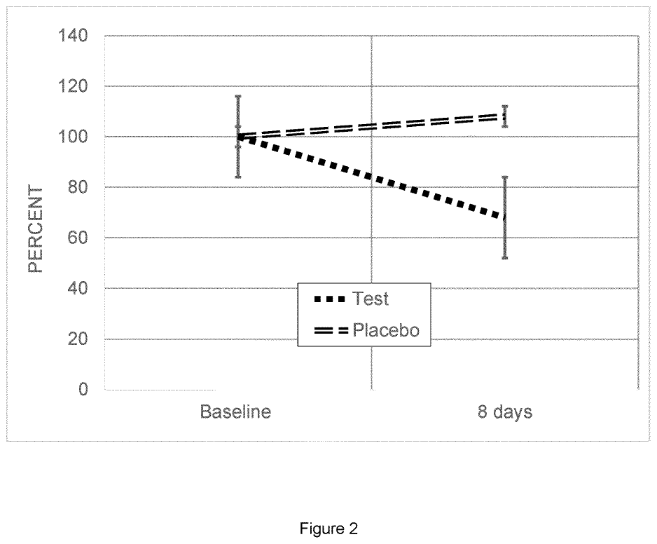

Subjects in the placebo group had a slightly higher mean OPI value at baseline but the difference was not statistically significant compared with the test group. After 8 days, a decrease was noted in the test group but not in the placebo group (p<0.05). The relative difference was 38% as illustrated in FIG. 2. Six patients in the test group and nine patients in the placebo group displayed bleeding on probing at baseline. The corresponding numbers after 8 days were three and nine patients, respectively. The inter-examiner test showed a 78% agreement of the OPI scores. Cohen's weighted Kappa was 0.734 (standard error 0.039), indicating a good agreement.

### Microbial Findings

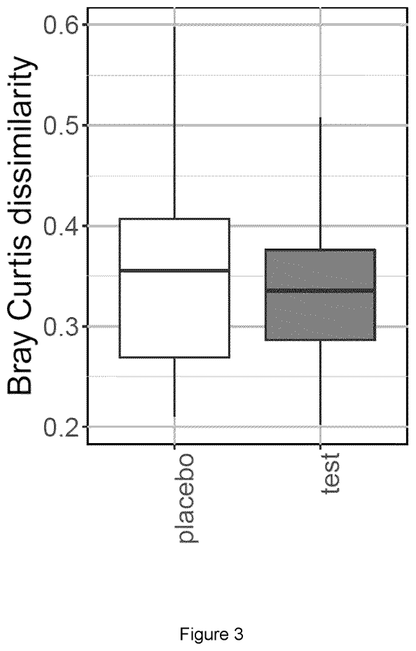

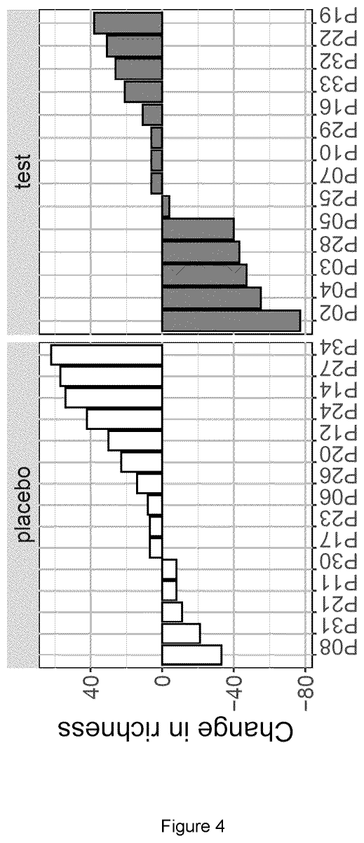

There was no difference in baseline microbial composition between the test and placebo group (p=0.317), and we found a consistent composition across time, with no statistical differences between the groups in the pairwise comparison of the global microbial composition at baseline and follow up (FIG. 3, p=0.704). There were no significant treatment effects on the microbial richness (FIG. 4).

### Side Effects:

One participant in the test group claimed nausea and “weird taste” and abandoned the project after the first day. Two participants did not like the taste of the mouthwash but used it as instructed. No other adverse events, or side effects, were reported to the investigators. All participants except one returned all the dispensed mouth rinse tubes to the clinic after eight days as agreed.

### Conclusion

Twice-daily use of a mouthwash containing Composition A decreased the amount of dental biofilm in adolescents with fixed orthodontic appliances over an 8-day period. The mean difference between the test group and the placebo group was 0.7 OPI units, which is of clinical relevance. The decreased amount of dental biofilm was obtained without significantly affecting the composition of the salivary microbiome.

### Example 5: Clinical Evaluation of the Effect of Composition a on Plaque Morphology in Healthy Individuals

The aim of the clinical study was to evaluate the effect of Composition A in a lozenge format on the amount and morphology of dental biofilm in healthy individuals.

### Materials and Methods

### Study Design and Ethical Approval:

The study applied a randomized double-blind placebo-controlled design with two parallel arms.

The trial was conducted at the Department of Dentistry and Oral Health at Aarhus University. The protocol was approved by the Central Jutland Regional Committee on Health Research Ethics (1-10-72-260-20).

### Experimental Product:

The experimental lozenge contained Composition A. The ingredients of the experimental lozenge and the placebo lozenge are described in Table 11. The placebo lozenge contained the same ingredients as the experimental lozenge except for the enzymes and had an identical taste, color and texture.

TABLE 11
Composition of experimental and placebo lozenges

| | Amount placebo | Amount exp. |
|---|---|---|
| Ingredient | (mg/tbl) | lozenge (mg/tbl) |
| Polyols | 1378.3 | 1378.3 |
| Silicon dioxide | 13.1 | 13.1 |
| Microcrystalline cellulose | 47.0 | 47.0 |
| Magnesium stearate | 22.5 | 22.5 |
| Steviol glycosides | 2.4 | 2.4 |
| DNase (SEQ ID NO: 1) | 0 | 18.8 |
| Mutanase (SEQ ID NO: 2) | 0 | 18.8 |
| Viscozyme ® L | 0 | 12.4 |
| Spirulina powder | 17 | 17 |
| Mint flavor | 19.7 | 19.7 |

### Power Calculation:

Due to the lack of previous clinical data with respect to the experimental lozenge, we anticipated that a mean difference of 0.5 units (SD 0.4) would be clinically meaningful, and an estimation with α=0.05 and β=1-0.8 indicated that 10 patients in each group needed to complete the study. To compensate for dropouts, 12 participants were enrolled in each arm.

### Study Population:

35 generally healthy males and females, at least 18 years of age, were assessed for eligibility, and 24 subjects were randomized to two study groups (n=12 in each group). Inclusion criteria were (1) generally healthy males and females 18 years of age, (2) able to read, sign and receive a copy of the signed informed consent form, and (3) have at least 20 natural teeth. Exclusion criteria were (1) clinically visible active caries lesions and/or periodontitis, (2) significant oral soft tissue pathology based on a visual examination, (3) history of allergy or significant adverse events following use of oral hygiene products such as toothpastes, mouth rinses, breath mints, lozenges, or chewing gum or their ingredients, (4) history of allergies to ingredients in the test product, (5) history of allergies towards enzymes, (6) self-reported as pregnant or nursing, (7) self-reported serious medical conditions, (8) antibiotic or anti-inflammatory medication within 30 days of screening visit, (9) orthodontic appliances, including retainers, peri/oral piercings, or removable partial dentures, and (10) acute sinusitis or severe oral-pharyngeal infections. We collected a signed consent from each subject. Eligible participants were randomly allocated to the test or placebo groups aided by computer software (www.randomization.com). The allocation concealment was guaranteed by an independent monitor not involved in data collection/analysis and was not revealed until all statistical calculations were finished. All subjects completed the study.

### Intervention:

The intervention period was 7 days (from day 1 to day 8).

At day 0, at day 1 (baseline), and after the intervention period, all participants received a professional tooth cleaning by a trained dentist after all indices were recorded and all samples taken.

At day 1, the participants were provided with tubes containing either placebo or experimental lozenges. The participants were instructed to take one lozenge three times daily, at least 30 min after a meal, for 7 days. The participants were told to place the lozenge on the dorsum of the tongue, and afterwards actively move it around the mouth to let it dissolve slowly. No normal oral hygiene procedures were allowed during the intervention period. The participants were instructed to return the used and (if any) unused tubes at day 8.

### Plaque and Saliva Sampling

At day 1 and day 8, 1 mL stimulated saliva was collected. At the same occasion, plaque was collected from the approximal space between the first and second molar of the third quadrant by scraping the tooth surface with a scaler. The samples were immediately transferred to sterile plastic vials, frozen to −80° C. and stored frozen until DNA-extraction and whole genome sequencing. The DNA extraction will be performed with Macherey-Nagel™ NucleoSpin™ Soil kit. The V3-V4 region of 16S will be PCR amplified using universal primers and sequenced using Illumina MiSeq 300 bp paired end. After analysis, the biological material will be destroyed.

### Plaque Assessment:

After 1, 2, and 8 days of intervention, plaque levels were assessed using the Turesky-modified Quigley-Hein Plaque Index (QHPI) (Quigley et al., JADA, 1962; 65:26-29; Turesky et al., J. Periodontol., 1970; 41:41-43).

At day 1, the Turesky-modified QHPI was assessed on the buccal surfaces of the first incisors, first premolars and first molars of the first and second quadrant to provide information on the individual level of plaque formation. Soft biofilm on the teeth was scored, and each surface was given a score from 0 to 5: score 0=no visible plaque; score 1=separate flecks of plaque at the cervical margin of the tooth; score 2=a thin, continuous band of plaque (up to one mm wide) at the cervical margin of the tooth; score 3=a band of plaque wider than one mm but covering less than one-third of the crown of the tooth; score 4=plaque covering at least one-third but less than two-thirds of the crown of the tooth; and score 5=plaque covering two-thirds or more of the crown of the tooth. Thereafter, the teeth were cleaned by a trained dentist.

At day 2 and 8, the modified QHPI was assessed on the buccal surfaces of the first incisors, first premolars and first molars of the first and second quadrant, respectively

### Statistical Methods:

Statistical analyses were performed using the software R. Age and gender differences between treatment groups were assessed by Mann-Whitney U Test and Fisher's Exact Test for Count Data, respectively. For comparisons of QHPI, mean values were calculated for day 1, day 2, and day 8. Differences between treatment groups at baseline (day 1) were assessed using the MannWhitney U Test. To estimate the treatment effect, differences between recordings at day 2 and 1 and day 8 and 1, respectively, were calculated. Normal distribution and homogeneity of variance of the data was assessed by Shapiro-Wilk Tests, qq-plots and F-tests, respectively. Two-sample t-tests were performed to assess the treatment effect on differences between baseline and day 2 or day 8. Moreover, linear regressions were performed with the differences between baseline and day 2 or day 8 as dependent variables, and baseline recordings and treatment type as explanatory variables. The level of significance was set to α=0.05.

### Saliva and Plaque Sampling and Analyses:

All samples were heat inactivated for 15 min at 95° C. followed by DNA extraction using the Macherey-Nagel™ NucleoSpin™ Soil kit according to the manufacturer's protocol. 200 μl saliva or the toothpicks used to sample the plaque were added directly into the beads tube. The V3-V4 region of 16S was PCR amplified using universal primers and sequenced using Illumina MiSeq 300 bp paired end. We included negative control samples (no material/DNA) as well as positive controls consisting of a known mock community to validate the analyses.

### Bioinformatics and Statistical Analysis of the Saliva and Plaque Samples:

We used the USEARCH pipeline to generate zero radius operational taxonomic units (OTUs; Edgar, Nat. Methods., 2013; 10:996-998) using the same setting as described by Larsen et al. (Gut Microbes, 2021, 13(1), 1988836). The taxonomic classification of the unique filtered amplicons was conducted with the QIIME feature-classifier classify-sklearn (Bolyen et al., Nat. Biotechnol. 2019, vol. 37, pp. 852-857) and the Human Oral Microbiome Database v15.22 (Chen et al., 2010). All samples were rarefied to 2000 reads per sample. For the global composition of the salivary microbiome at baseline, we applied the Adonis Permanova test on the dissimilarity matrix using the Bray-Curtis metric (Bray and Curtis, Monographs, 1957; 27:326-349). To investigate if the lozenge introduced a change in the global microbiome composition overtime, the Bray-Curtis dissimilarity between baseline and follow-up samples for each individual was tested using a paired t-test. We established a linear mixed model to test effects of the mouthwash compared to the placebo group on microbial richness from baseline to follow up. The null hypothesis was that the change in richness between baseline and follow up for the test group was equal to the change in the placebo group. We used a similar model to test for significant changes for all OTUs present in more than 32 samples, where each OTU count was log transformed. We corrected all p-values for multiple testing according to Benjamini and Hochberg (J R Stat Soc Series, B. 1995; 57:289-300).

### Results

### Baseline Data:

The mean age was 30.0 years in the test group and 29.7 years in the placebo group. The gender distribution (n female/n male) was 3/9 in the test group and 7/5 in the placebo group. There was no difference in Turesky-modified QHPI between the test group and the placebo group at baseline.

### Clinical Findings:

Differences in plaque recordings between day 2 and baseline were significantly lower for the test group compared to the placebo group (−0.82±0.74 SD vs −0.09±0.45 SD; p=0.01 after removal of an outlier from each group).

Differences in plaque recordings between day 8 and baseline appeared slightly lower for the test group compared to the placebo group, but the difference was not statistically significant (1.03±1.42 SD vs 1.61±1.36 SD; p=0.39). However, the examiner noted profound difference in plaque morphology between the test group and the placebo group at day 8. Visual inspection by the examiner (a trained dentist) indicated that the majority of subjects in the test group had thin plaque (86% vs. 14%), whereas the majority of subjects in the placebo group had thick plaque (56% vs. 44%).

Importantly, differences in plaque morphology are not taken into account by the Turesky-modified QHPI, and the total amount of dental biofilm is expected to be lower in subjects having thin plaque morphology compared to subjects having thick plaque morphology.

### Microbial Findings:

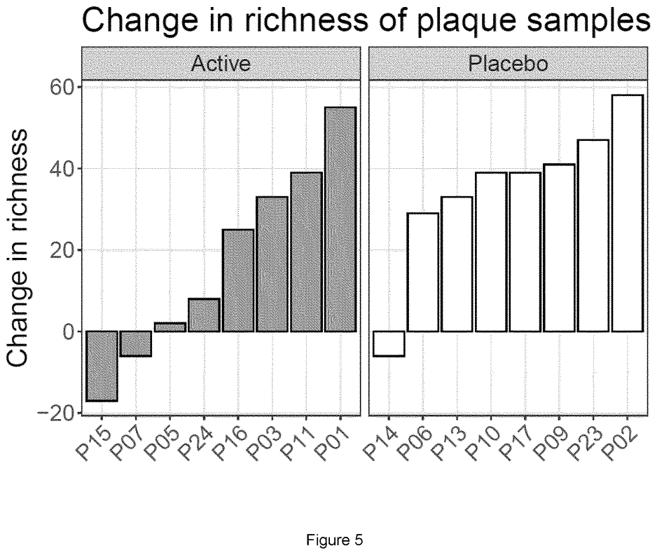

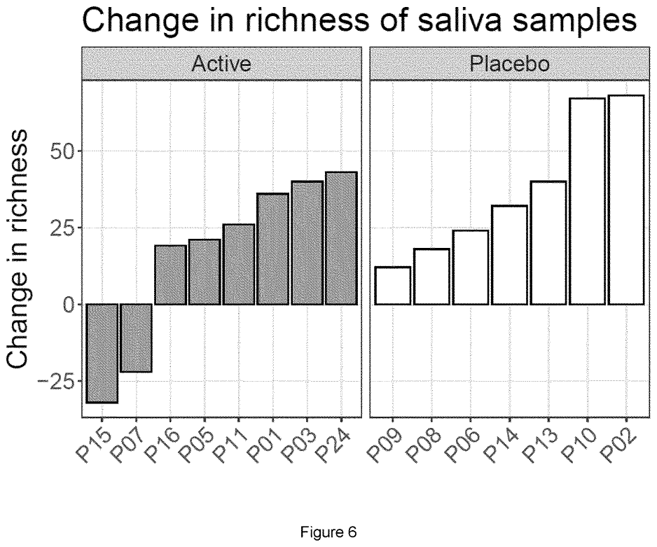

A higher increase in microbial richness between baseline (day 1) and at day 8 was observed for the placebo group compared to the treatment group. This was statistically significant for the plaque samples (FIG. 5, p=0.04) and a similar tendency was observed for the saliva samples (FIG. 6, p=0.12).

### Conclusion

Thrice-daily use of a lozenge containing Composition A decreased the amount of dental biofilm in healthy individuals over a 7-day period. The decrease was statistically significant at day 2 (i.e., after 1 day of treatment) and remained slightly lower at day 8. Visual inspection at day 8 revealed that plaque morphology was different in the test group compared to the placebo group. Without being bound by theory, it is speculated that the thin plaque morphology observed in the test group results in the dental biofilm being more susceptible to mechanical abrasion. Analysis of the saliva and plaque microbiome revealed that the increase in microbial richness was higher for the placebo group compared to the treatment group at day 8.

### Example 6: Live/Dead Baclight™ Bacterial Viability Fluorescence assay on Human Saliva Biofilm Following Exposure to Composition A

The Live/Dead Baclight™ Bacterial Viability kit utilizes a mixture of SYTO 9 and propidium iodide stains to quantitatively distinguish live and dead bacteria in a biofilm population containing a mixed range of bacterial types. Thus, bacteria with intact cell membranes stain fluorescent green, whereas bacteria with damaged membranes stain fluorescent red.

### Biofilm Removal Assay

A biofilm removal assay was carried out using human saliva biofilm grown in 96-well microtiter plates (black/clear bottom plates, ThermoFisher Scientific™ 265301) according to the method described in WO 2020/099490 with some modifications. Briefly, samples containing biofilm grown for 24 h were treated with either 200 μL citrate phosphate buffer, pH 6 (McIlvaine buffer, prepared by mixing 12.63 ml 0.2 M Na₂HPO₄ and 7.37 ml 0.1 M citric acid) 6 as control, or the following treatment solutions:
Composition A in McIlvaine buffer, pH 6
Chlorhexidine 0.2% (Apotekernes A.m.b.a., Mileparken 20E 2740 Skovlunde, Denmark)
Ethanol 96% (VWR International)
Benzalkonium chloride 0.1% (alkylbenzyldimethylammonium chloride, CAS no. 63449-41-2, Sigma-Aldrich)
Solimo® mouthwash (Amazon)
Colgate® Plax cool mint mouthwash (Colgate-Palmolive)

Samples were incubated at 37° C. under agitation (50 rpm) for 1 min or 5 min. Control and treatment samples were carried out in 8 replicates/plate.

After treatment, samples were rinsed twice with 200 μL 0.9% NaCl and stained with Live/Dead Baclight™ Bacterial Viability kit (Invitrogen, #L7012) following the manufacture's protocol.

### Preparation of Dyes

For one 96-well plate, a 1:1 mixture was prepared by dissolving 30 μL of SYTO 9 (3.34 mM), and 30 μL of propidium iodide (20 mM) in 10 mL MilliQ water. 100 μL of dye solution was dispensed to each well, and the 96-well plates were incubated at room temperature in the dark for 30 minutes under agitation (50 rpm). Samples were rinsed twice with 0.9% NaCl and fluorescence measurements were carried out using a Tecan reader Infinite M1000 PRO.

### Fluorescence Intensity Measurements

The excitation/emission for SYTO 9 was 485/530 (emission 1, abbreviated em1; green) and for propidium iodide was 485/630 (emission 2, abbreviated em2; red) for each well of the entire plate.

### Data Analysis

The fluorescence intensity of the stained bacteria (F_{cell}) at emission 1 (green) was divided by the fluorescence intensity at emission 2 (red) to obtain the ratio of live cells versus dead cells (Ratio G/R):

$${Ratio}_{G / R} = {F}_{cell} em 1 / {F}_{cell} em 2$$

TABLE 12
Ratio_{G/R} after 1 min and 5 min

| | |
|---|---|
| 5 min | 1 min |

| Treatment | Mean | Std Err | Mean | Std Err |
|---|---|---|---|---|
| Control (buffer) | 2.3 | 0.04 | 2.29 | 0.05 |
| Composition A | 2.27 | 0.06 | 2.36 | 0.05 |
| Chlorhexidine 0.2% | 0.58 | 0.03 | 0.53 | 0.02 |
| Ehanol 96% | 0.3 | 0.01 | 0.3 | 0.01 |
| Benzalkonium 0.1% | 0.29 | 0.03 | 0.18 | 0.02 |
| Solimo mouthwash | 0.24 | 0 | 0.26 | 0.01 |
| Colgate Plax mouthwash | 0.89 | 0.09 | 0.55 | 0.08 |

The ratio of green to red fluorescence emission is proportional to the relative numbers of live bacteria. The data listed in Table 12 show that treatment with Composition A resulted in a live:dead ratio similar to that of the control treatment and higher than the live:dead ratio obtained following treatment with a number of commercially available biocidal/antiseptic solutions such as chlorhexidine, benzalkonium, ethanol and the mouthwash products Solimo and Colgate Plax.

These results of the Live/Dead Baclight assay clearly indicate that Composition A does not affect the live/dead ratio of bacteria found in a biofilm grown from human saliva, and this is in agreement with the clinical findings that treatment with Composition A does not alter the oral microbiome.

### Example 7: Canine Multispecies Biofilm Prevention Assay

The biofilm prevention effect of the composition of the invention comprising a DNase, a mutanase, and a beta-glucanase was tested on a multispecies biofilm grown on 96-well microtiter plates consisting of canine dental pathogens.

The composition was tested on multispecies biofilm grown consisting of five canine dental pathogens: Neisseria zoodegmatis DSM21643, Pasteurella canis DSM22968, Pseudomonas fragi DSM3456, Capnocytophaga canis DSM101831, and Enterococcus faecalis DSM20478

96-well microtiter plates (Nunclon Delta surface, ThermoFisher Scientific #167008) were filled with 75 μl of Tripticase Soy Broth (TSB) containing 1×10⁷ CFU/ml bacterial inoculum of each of the five strains mentioned above and 25 μl of enzyme solution in 25 mM HEPES buffer (pH 7) to yield a final concentration of 60 ppm. For control samples the enzyme solution was replaced with 25 mM HEPES buffer (pH 7). Plates were incubated at 30° C. for 24 hours without shaking in a ThermoFisher Scientific™ Rectangular AnaeroBox™ Container under microaerophilic conditions (ThermoFisher Scientific™ AnaeroGen 2.5L #AN0025A). Enzyme and control samples were carried out with 8 replicates.

After incubation, planktonic bacteria were removed by two gentle washes with 100 μl 0.9% sodium chloride and biofilms were stained with 0.095% crystal violet solution for 15 min at room temperature. Plates were rinsed twice with 100 μL 0.9% sodium chloride and adhered dye was dissolved with a solution of 96% ethanol and 0.1% acetic acid in water. Absorbance was measured at 600 nm with a microplate reader SpectraMax M3, Molecular Devices.

For the data processing, the absorbance was taken to be proportional to the extent of remaining biofilm after enzyme or buffer treatment. The results were expressed as percentage of biofilm prevention and was calculated as follows:

100−((A600 nm enzyme treated sample)/(A600 nm buffer control treated sample)×100)

where A600 nm refers to the average of eight absorbances measured at 600 nm of either enzyme or buffer treated samples. Results are listed in Table 13 below:

TABLE 13
Prevention of canine multispecies

| | Biofilm | |
|---|---|---|
| Enzyme composition | prevention (%) | Std Err |
| Composition A | 82.9 | 0.6 |

Results showed that the Composition A provides an 82.9% prevention effect on formation of canine multispecies biofilm.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1: An enzymatic oral care composition comprising a DNase, a mutanase, a beta-glucanase, and at least one oral care ingredient.

2: The oral care composition according to claim 1, wherein the DNase is a polypeptide having DNase activity and a sequence identity of at least 60% to SEQ ID NO:1.

3: The oral care composition according to claim 1, wherein the mutanase is selected from the group consisting of:
a) a polypeptide having endo-1,3-α-glucanase activity and a sequence identity of at least 60% to the polypeptide of SEQ ID NO: 2;
b) a polypeptide having endo-1,3-α-glucanase activity and a sequence identity of at least 60% to the polypeptide of SEQ ID NO: 4;
c) a polypeptide having endo-1,3-α-glucanase activity and a sequence identity of at least 60% to the polypeptide of SEQ ID NO: 5;
d) a polypeptide having endo-1,3-α-glucanase activity and a sequence identity of at least 60% to the polypeptide of SEQ ID NO: 6;
e) a polypeptide having endo-1,3-α-glucanase activity and a sequence identity of at least 60% to the polypeptide of SEQ ID NO: 7;
f) a polypeptide having endo-1,3-α-glucanase activity and a sequence identity of at least 60% to the polypeptide of SEQ ID NO: 8;
g) a polypeptide having endo-1,3-α-glucanase activity and a sequence identity of at least 60% to the polypeptide of SEQ ID NO: 9;
h) a polypeptide having endo-1,3-α-glucanase activity and a sequence identity of at least 60% to the polypeptide of SEQ ID NO: 10;
i) a polypeptide having endo-1,3-α-glucanase activity and a sequence identity of at least 60% to the polypeptide of SEQ ID NO: 11;
j) a polypeptide having endo-1,3-α-glucanase activity and a sequence identity of at least 60% to the polypeptide of SEQ ID NO: 12;
k) a polypeptide having endo-1,3-α-glucanase activity and a sequence identity of at least 60% to the polypeptide of SEQ ID NO: 13; and
l) a polypeptide having endo-1,3-α-glucanase activity and a sequence identity of at least 60% to the polypeptide of SEQ ID NO: 14.

4: The oral care composition according to claim 1, wherein the beta-glucanase is selected from the group consisting of:
a) a polypeptide having endo-1,3(4)-β-glucanase activity and a sequence identity of at least 60% to the polypeptide of SEQ ID NO: 3;
b) a polypeptide having endo-1,3(4)-β-glucanase activity and a sequence identity of at least 60% to the polypeptide of SEQ ID NO: 15; and
c) a polypeptide having endo-1,3(4)-β-glucanase activity and a sequence identity of at least 60% to the polypeptide of SEQ ID NO: 16.

5: The oral compositions according to claim 1,
i) wherein the DNase is a polypeptide having DNase activity and a sequence identity of at least 60% to SEQ ID NO: 1;
ii) wherein the mutanase is a polypeptide having endo-1,3-α-glucanase activity and a sequence identity of at least 60% to the polypeptide of SEQ ID NO: 2; and
iii) wherein the beta-glucanase is selected from the group consisting of:
a) a polypeptide having endo-1,3(4)-β-glucanase activity and a sequence identity of at least 60% to the polypeptide of SEQ ID NO: 3;
b) a polypeptide having endo-1,3(4)-β-glucanase activity and a sequence identity of at least 60% to the polypeptide of SEQ ID NO: 15; and
c) a polypeptide having endo-1,3(4)-β-glucanase activity and a sequence identity of at least 60% to the polypeptide of SEQ ID NO: 16.

6: The oral care composition according to claim 1,
i) wherein the DNase comprises, consists essentially of, or consists of SEQ ID NO: 1;
ii) wherein the mutanase comprises, consists essentially of, or consists of SEQ ID NO: 2; and
iii) wherein the beta-glucanase comprises, consists essentially of, or consists of SEQ ID NO: 3 and/or SEQ ID NO: 15 or SEQ ID NO: 16.

7: The oral care composition according to claim 6,
i) wherein the DNase comprises, consists essentially of, or consists of SEQ ID NO: 1;
ii) wherein the mutanase comprises, consists essentially of, or consists of SEQ ID NO: 2; and
iii) wherein the beta-glucanase comprises, consists essentially of, or consists of SEQ ID NO: 3.

8: The oral care composition according to claim 1, wherein at least one of the DNase, mutanase, and beta-glucanase has on par or improved chemical stability in the presence of at least one oral care ingredient selected from the group consisting of benzoate, EDTA, ethanol, glycerol, phosphate, fluoride, hydrogen peroxide, and mannitol.

9: The oral care composition according to claim 8, wherein chemical stability is determined according to Example 1 herein and wherein chemical stability is determined as thermal stability defined by the thermal unfolding transition midpoint (Tm) in the presence of a particular oral care ingredient.

10: The oral care compositions according to claim 1, wherein the oral care composition provides improved biofilm prevention compared to Composition B as determined according to Example 2 herein.

11: The oral care composition according to claim 1 in the form of an internal oral care composition in the form of a toothpaste or toothpaste tablet, dental cream, mouthwash or mouthwash tablet, mouth rinse, lozenge, pastille, chewing gum, confectionary, or candy.

12: The oral care composition according to claim 1 in the form of an external oral care composition in the form of denture cleaning solution, denture cleaning tablet, or denture cleaning powder.

13-14. (canceled)

15: A kit of parts comprising:
a) an oral care composition according to claim 1; and
b) instructions for use.
